(19) United States
(12) Patent Application Publication
(10) Pub. No.: US 2023/0106721 A1
(43) Pub. Date: Apr. 6, 2023

(54) Protection of Next-Generation Probiotics during Processing

(71) Applicant: Massachusetts Institute of Technology, Cambridge, MA (US)

(72) Inventors: Chelsea Catania, Somerville, MA (US); Ariel Furst, Cambridge, MA (US); Gang Fan, Jamaica Plain, MA (US); Pris Wasuwanich, Orlando, FL (US)

(21) Appl. No.: 17/817,710

(22) Filed: Aug. 5, 2022

Related U.S. Application Data
(60) Provisional application No. 63/252,494, filed on Oct. 5, 2021.

(51) Int. Cl. A61K 35/742; A23L 33/135; A61K 9/00; A61K 47/52; A61K 47/69

(52) U.S. Cl. CPC A61K 35/742; A23L 33/135; A61K 9/0053; A61K 47/52; A61K 47/6903

(57) ABSTRACT

Prokaryotic cells having a metal-phenolic network coating are disclosed, as are compositions including such cells, methods for their use, and methods for producing such cells.

### CROSS REFERENCE

This application claims priority to U.S. Provisional Patent Application Ser. No. 63/252,494 filed Oct. 5, 2021, incorporated by reference herein in its entirety.

### FEDERAL FUNDING STATEMENT

This invention was made with Government support under Grant No. IIP2037748 awarded by the National Science Foundation. The Government has certain rights in the invention.

### BACKGROUND

The gut microbiome is essential to maintain overall health and prevent disease, which can occur when these microbes are not in homeostasis. Microbial biotherapeutics are important to combat these issues, but they must be alive at the time of de-livery for efficacy. Many potentially therapeutic species are anaerobes and difficult to manufacture, making their production nearly impossible.

### SUMMARY

In one aspect, the disclosure provides prokaryotic cells having a coating comprising metal-phenolic networks (MPN). In one embodiment, the coating comprises a complete coating over the entire cell surface. In various embodiments, the coating comprises a single MPN layer, or comprises 2, 3, 4, 5, 6, 7, 8, 9, 10, or more MPN layers. In one embodiment, the coating is between about 10 nm and about 100 nm in thickness. In various embodiments, the coating comprises a single metal ion component and a single phenolic component, or the coating comprises more than one metal ion component and/or more than one single phenolic component. In some embodiments, the metal ion component in the MPN comprises one or more cations of aluminum (Al), vanadium (V), chromium (Cr), manganese (Mn), iron (Fe), cobalt (Co), nickel (Ni), copper (Cu), zinc (Zn), zirconium (Zr), molybdenum (Mo), ruthenium (Ru), rhodium (Rh), cadmium (Cd), cerium (Ce), europium (Eu), gadolinium (Gd), terbium (Tb), or combinations thereof, including but not limited to one or more of Fe³⁺, Cr²⁺, Cr³⁺, Cr⁶⁺, Cu⁺, Cu²⁺, Cu³⁺, Cu⁴⁺, Mn²⁺, Mn³⁺, Mn⁴⁺, Mn⁶⁺, Mn⁷⁺, Mo²⁺, Mo³⁺, Mo⁴⁺, Mo⁶⁺, Co²⁺, Ni²⁺, Cd²⁺, Al³⁺, V³⁺, Rh³⁺, Ru³⁺, Zr⁴⁺, Eu³⁺, Gd³⁺, Tb³⁺. and/or Zn²⁺. In one embodiment, the metal ion component in the MPN comprises an iron cation, such as comprises Fe³⁺. In other embodiments, the polyphenol component of the MPNs comprises one or more of flavonoids (including isoflavonoids and neoflavonoids), tannins, condensed tannins, phenolic acids, catechols, lignans, and stilbenes. In a further embodiment, the polyphenol component of the MPN comprises tannic acid (TA), gallic acid (GA), epigallocatechin gallate (EGCG), or combinations thereof. In another embodiment, the MPNs comprise an iron cation, such as Fe³⁺, complexed with one or more of tannic acid (TA), gallic acid (GA), and epigallocatechin gallate (EGCG). In other embodiments, the cell is a bacterial cell, an archaeal cell, or an anaerobic cell. In some embodiments, the prokaryotic cell is selected from the group consisting of Bacteroides species including but not limited to Bacteroides thetaiotaomicron, Bacillus species, including but not limited to Bacillus subtilis. Bacillus Calmette-Guerin (BCG) or Mycobacterium smegmatis. In one embodiment, the cell is lyophilized. In another embodiment, the cells further comprise one or more functional groups bound to the MPNs.

In another embodiment, the disclosure provides compositions comprising a plurality of the prokaryotic cells of any embodiment or combination of embodiments of the disclosure. In one embodiment, the plurality of prokaryotic cells are lyophilized. In another embodiment, the composition does not include a cryoprotectant. In a further embodiment, the composition is formulated for oral administration. In other embodiments, the formulation comprises beads, powders, capsules, tablets, drops, oil suspensions, gels, lozenges, and/or liquid suspensions. In some embodiments, the composition is present in a food or beverage product, including but not limited to milk, yogurt, cheese, cream, chocolate, meat, chewing gum, kombucha or other fermentation products, and animal feed or drinking water.

In another aspect, the disclosure provides methods for treating a disorder, comprising administering to a subject in need thereof an amount effective of the non-pathogenic prokaryotic cells and/or the compositions of any previous claim to treat the disorder. In one embodiment, the non-pathogenic prokaryotic cells and/or compositions are administered orally. In another embodiment, the disorder comprises non-muscle invasive bladder cancer (NMIBC), and wherein the non-pathogenic prokaryotic cells and/or the composition comprises Bacillus Calmette-Guerin (BCG). In a further aspect, the disclosure provides methods for forming a coating of MPNs on prokaryotic cells, comprising adding metal ions and one or more polyphenols to a suspension of prokaryotic cells, and adjusting pH of the suspension to a final pH of between 7.0 and 8.0 by addition of 3-(N-morpholino) propanesulfonic acid (MOPS) buffer. In one embodiment, the method is initiated under nitrogen atmosphere.

### DESCRIPTION OF THE FIGURES

FIG. 1(a-b). Metal-phenolic network (MPN) assembly on microbes. (a) Cells were coated with a mixture of polyphenols and metal ions. Three polyphenols were evaluated: GA, EGCG, and TA. (b) UV-vis spectra of MPN-encapsulated E. coli.

FIG. 2(a-b). Fluorescence and SEM images of encapsulated E. coli. (a) Cells were treated with Alexa-Fluor 647 BSA, which adheres to MPNs but not uncoated cells. Fluorescence images of (i-iv) uncoated and (v-viii) MPN coated E. coli (25922GFP) in the phase contrast channel, FITC channel (GFP), Cy5 channel (Alexa-Fluor 647 BSA), and merged (scale bar: 20 μm). All fluorescence is represented as “white” in the images. (b) SEM images of (i-ii) MPN coated and (iii-iv) uncoated E. coli (MG1655). (i, iii) Scale bar 1 μm, (ii) scale bar: 100 nm, (iv) scale bar: 200 nm.

FIG. 3(a-d). Microbial growth following encapsulation and dis-solution. (a) CFUs for coated E. coli. (b) CFUs for coated E. coli after acid treatment. (c) Growth curves (based on A600) of E. coli alone, with iron only, with TA only, and MPN-coated. Although there is a growth lag for MPN-coated cells, no change in the exponential growth rate is observed. (d) Growth curves of uncoated B. thetaiotaomicron after dilute AA treatment (5 min). Error bar represents the standard deviation of n=3 replicates.

FIG. 4(a-b). MPN protection of microbes during lyophilization. (a) OD600 of lyophilized E. coli and B. thetaiotaomicron following 48 h growth in the presence and absence of MPNs and cryoprotectants; (b) Growth curves of B. thetaiotaomicron following lyophilization without cryoprotectants (PC: phosphate-citrate buffer; PB: phosphate-buffered saline). Error bar represents the standard deviation of n=3 replicates.

FIG. 5. UV-vis spectra of E. coli (MG1655) coated in MPNs comprised of different phenols in combination with ferric cations (Fe³⁺), as well as uncoated E. coli. (GA—gallic acid, ECGC—Epigallocatechin gallate, TA—Tannic acid).

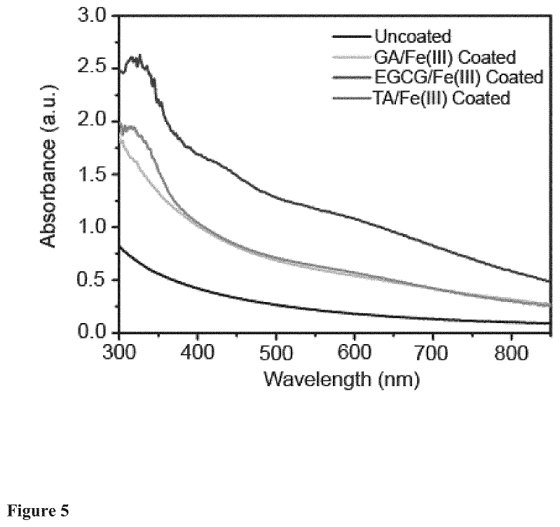

FIG. 6. CFUs counting for MPN-coated E. coli (DH10β) before and after ascorbic acid treatment. The control represents uncoated E. coli. (Error bars represent SD for n=3 replicates)

FIG. 7(a-c). Growth curves (based on absorbance at 600 nm) of E. coli (DH10β) alone, MPN-coated E. coli [a) GA-Fe³⁺; b) EGCG-Fe³⁺; c) TA-Fe³⁺.] and ascorbic-acid (AA)-treated (1 h) MPN-coated E. coli.

FIG. 8(a-b). Growth curves of (a) MPN-coated B. thetaiotaomicron after different concentrations of ascorbic acid (10 mM (black), 1 mM (light grey) and 0.1 mM (dark grey)) treatment and (b) MPN-coated B. thetaiotaomicron at different coating thickness (For 0.1× condition, both tannic acid and iron chloride concentration were decreased 10× to 0.16 mg mL⁻¹ and 0.024 mg mL⁻¹, respectively). Error bars represents SD for n=3 replicates.

FIG. 9(a-f). Fluorescence images of E. coli following MPN encapsulation. Cells were treated with non-fluorescent fluorescein diacetate (FDA) to measure enzymatic activity and cell-membrane integrity (turn-on fluorescence). Fluorescence images of (a-c) uncoated and (d-f) MPN-coated E. coli (DH10β) in (a, d) phase contrast channel, (b, e) FITC channel (GFP), and (c,f) merged channel (Scale bar: 20 μm; all fluorescence represented as white).

FIG. 10. Growth curves monitored at OD₆₀₀ of MPN-coated and uncoated B. thetaiotaomicron following lyophilization with cryoprotectants (PC: phosphate-citrate buffer; PB: phosphate-buffered saline; cryoprotectant: 0.1 mM trehalose). Error bars represents SD for n=3 replicates.

FIG. 11(a-b). Growth curves monitored at OD₆₀₀ of MPN-coated and uncoated B. thetaiotaomicron following (a) 1 h and (b) 2 h oxygen exposure [ascorbic acid] 10 mM. Error bars represent SD for n=3 replicates.

FIG. 12(a-c). Growth curves monitored at OD₆₀₀ of B. thetaiotaomicron alone, with iron only, with tannic acid (TA) only, and with both (MPN-coated) following lyophilization. Error bars represent SD for n=3 replicates (a-c).

FIG. 13. OD₆₀₀ of lyophilized E. coli (DH10β) following 48 h growth in the presence or absence of MPNs, FeCl₃, tannic acid (TA) and cryoprotectants (PC: phosphate-citrate buffer; PB: phosphate-buffered saline; T: 0.1 mM trehalose).

FIG. 14. Growth curves of E. coli (DH10β) alone, with iron only, with TA only, and with both (MPN-coated) following lyophilization. Shaded region represents error bars represent SD for n=3 replicates.

FIG. 15(a-d): Abiotic characterization of metal-phenolic networks (MPNs). a) Polyphenol components used in the MPN assemblies. b) Example chelation of metal ions (Fe³⁺ and Zn²⁺) to polyphenols. c) UV-Vis absorbance of Fe³⁺-TA, Fe³⁺-GA, and Fe³⁺-EGCG complexes in MOPS buffer (10 mM, pH 7.5). d) Fe³⁺-TA, Fe³⁺-GA, and Fe³⁺-EGCG complexes formed on the surface of silica particles (1 mg/mL).

FIG. 16(a-b). Fluorescence and TEM images of encapsulated B. subtilis. (a) Cells were treated with live/dead staining kit. Shown are fluorescence images of (i-iii) uncoated and (iv-vi) Fe³⁺-TA-coated B. subtilis in the FITC channel (CYTO-9), and TxRed channel (propidium iodide) and the merged images. Scale bars indicate 20 μm, and all fluorescence appears as white. (b) TEM images of (vii, viii) uncoated and (ix, x) Fe³⁺-TA-coated B. subtilis.

FIG. 17(a-b). Fe³⁺-phenols encapsulation protect microbes during lyophilization. (a) OD₆₀₀ of lyophilized B. subtilis following 48 h of growth in the presence and absence of Fe³⁺-phenols and cryoprotectants (0.1M) (PC, phosphate citrate buffer; PB, phosphate-buffered saline). (b) Growth curves of Fe³⁺-phenols-coated and uncoated B. subtilis following lyophilization without cryoprotectants. Shaded region represents error bars represent SD for n=3 replicates.

FIG. 18(a-b). Metal-GA encapsulation protect microbes during lyophilization. (a) OD₆₀₀ of lyophilized B. subtilis following 48 h of growth in the presence and absence of metal-GA and cryoprotectants (0.1M) (PC, phosphate citrate buffer; PB, phosphate-buffered saline). (b) Growth curves of Fe³⁺-TA, Fe³⁺-GA-coated and uncoated lyophilized B. subtilis following dilute AA treatment (5 min). Shaded region represents error bars represent SD for n=3 replicates.

FIG. 19(a-f). UV-vis spectra of metal-phenolic networks. UV-Vis absorbance of (a) Al³⁺-TA, Al³⁺-GA, and Al³⁺-EGCG complexes in MOPS buffer (10 mM, pH 7.5), (b) Al³⁺-TA, Al³⁺-GA, and Al³⁺-EGCG complexes after addition of silica particles (1 mg/mL); (c) Mn²⁺-TA, Mn²⁺-GA, and Mn²⁺-EGCG complexes in MOPS buffer (10 mM, pH 7.5), (d) Mn²⁺-TA, Mn²⁺-GA, and Mn²⁺-EGCG complexes after addition of silica particles (1 mg/mL); (e) Zn²⁺-TA, Zn²⁺-GA, and Zn²⁺-EGCG complexes in MOPS buffer (10 mM, pH 7.5), (f) Zn²⁺-TA, Zn²⁺-GA, and Zn²⁺-EGCG complexes after addition of silica particles (1 mg/mL).

FIG. 20. Growth curves monitored at OD₆₀₀ of B. subtilis alone and MPN-coated B. subtilis (“2×” means coating is repeated twice).

FIG. 21. Bacterial viability assessment shows live/dead B. subtilis after Fe³⁺-TA encapsulation without addition of MOPS buffer (Viability: 54%). Representative composite images of DIC, live and dead fluorescence channels for B. subtilis.

FIG. 22(a-b). Growth curves monitored at OD₆₀₀ of Fe³⁺-polyphenol-coated and uncoated B. subtilis following lyophilization with cryoprotectants (a) PB: phosphate-buffered saline; (b) PC: phosphate-citrate buffer; cryoprotectant: 0.1 M trehalose). Shaded region represents error bars represent SD for n=3 replicates.

FIG. 23(a-c). Growth curves monitored at OD₆₀₀ of metal-GA-coated and uncoated B. subtilis following lyophilization with or without cryoprotectants ((a) PC: phosphate-citrate buffer; (b) PCT: phosphate-citrate buffer; cryoprotectant: 0.1 M trehalose); (c) PBT Buffer: phosphate-buffered saline; cryoprotectant: 0.1 M trehalose. Shaded region represents error bars represent SD for n=3 replicates.

FIG. 24(a-b). Growth curves (a-b) monitored at OD₆₀₀ of B. subtilis alone, with metals (Fe³⁺, Zn²⁺, and Al³⁺) only, with polyphenols (TA, GA) only, and with both (Fe-TA, Fe-GA-MPN-coated) following lyophilization in PC buffer. Shaded region represents error bars represent SD for n=3 replicates.

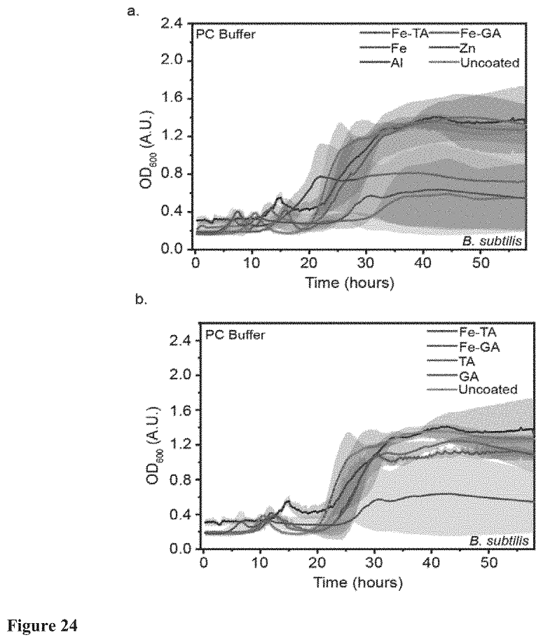

FIG. 25(a-b). Growth curves (a-b) monitored at OD₆₀₀ of B. subtilis alone, with metals (Fe³⁺, Zn²⁺, and Al³⁺) only, with polyphenols (TA, GA) only, and with both (Fe-TA, Fe-GA-MPN-coated) following lyophilization in PC buffer supplement with 0.1 M trehalose. Shaded region represents error bars represent SD for n=3 replicates.

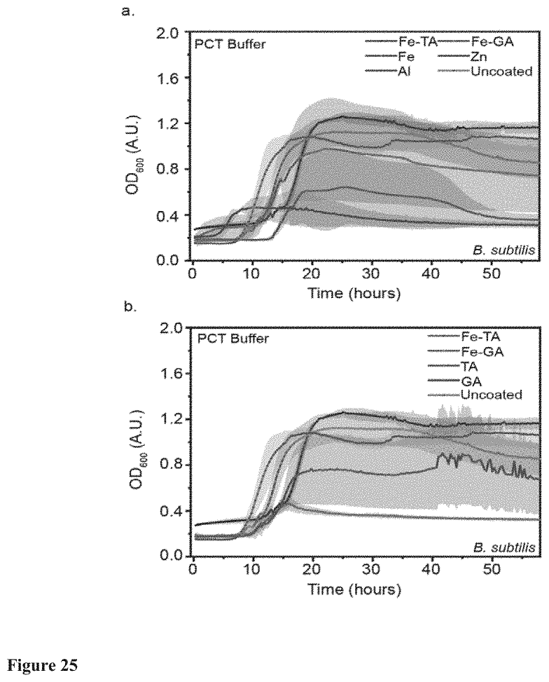

FIG. 26: Growth curves monitored at OD₆₀₀ of Fe³⁺-polyphenol-coated and uncoated M. smegmatis following lyophilization and encapsulation in biodegradable polymers (PLGA-PEG-PLGA-Poly(lactide co-glycolide) block poly(ethylene glycol) block poly(lactide co-glycolide)). The MPN-coated cells recover significantly faster than uncoated.

FIG. 27: (a-b). Mycobacteria. (a) LMCT band for MPN-coated M. smegmatis but not for the negative controls.

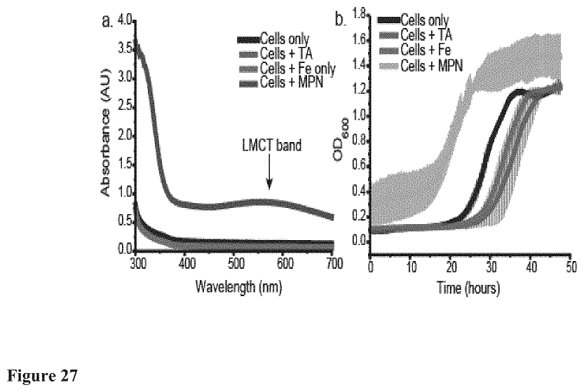

FIG. 28(a-b). Protection of P. chlororaphis. (a) Viability of TA/Fe(III) cells at temperatures ranging from 4° C. to 30° C. (b) OD₆₀₀ of coated and uncoated cells following freeze-drying and storage at 50° C. for 48 h.

FIG. 29. Protection of P. chlororaphis from heat and humidity. Conditions were: cells were lyophilized then stored at 50° C. for 48 h under humid conditions (minimum of 60%). TA—Tannic Acid; GA—gallic acid; EGCG—Epigallocatechin gallate.

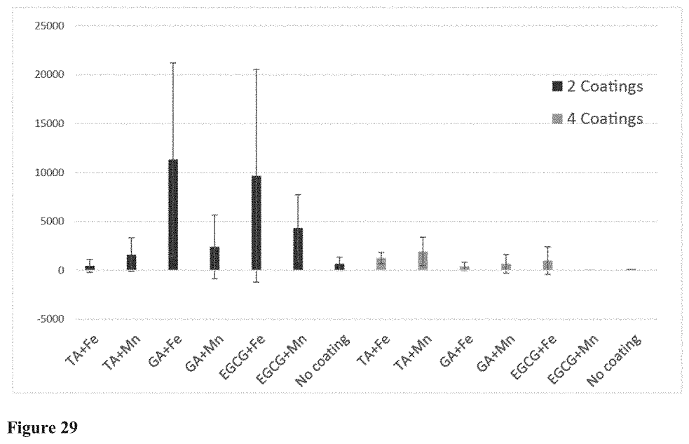

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All embodiments of any aspect of the invention can be used in combination, unless the context clearly dictates otherwise.

As used herein, “about” means +/−5% of the recited value.

In a first aspect, the disclosure provides prokaryotic cell having a coating comprising metal-phenolic networks.

As used herein, metal-phenolic networks (MPNs) are non-covalent coordination complexes of metal ions and polyphenols. MPNs adsorb to surfaces through noncovalent interactions. Each cell is coated (sometimes referred to as “encapsulated”) with an assembled metal-phenolic network.

As used herein, “phenol” is an aromatic organic compound with the molecular formula C₆H₅OH. Phenol as used herein also includes benzenediol (molecular formula C₆H₄(OH)₂) and benzenetriol (molecular formula C₆H₃(OH)₃) groups.

As used herein, polyphenols are organic compounds characterized by one or more phenol units, provided at least 2, 3, or more hydroxyl groups are present. In some embodiments, the polyphenol comprises two or more phenol groups, which can independently be phenol, benzenediol, or benzenetriol groups. For example, in certain embodiments, polyphenol independently includes two or more benzenediol and/or benzenetriol groups.

In one embodiment, the coating comprises a complete coating over the entire cell surface (also referred to as contiguous herein). In other embodiments, the coating may be an incomplete coating over the cell surface. In one embodiment, the coating may comprise a single MPN layer. In other embodiments, the coating comprises 2, 3, 4, 5, 6, 7, 8, 9, 10, or more MPN layers. As discussed in the accompanying examples, each layer is approximately 10 nm thick, and thus the thickness of the coating may be readily adjusted by coating with a number of MPN layers as deemed suitable for an intended purpose.

In other embodiments, the coating is between about 10 nm and about 100 nm in thickness. In various other embodiments, the coating is between about 10 nm and about 90 nm, about 10 nm and about 80 nm, about 10 nm and about 70 nm, about 10 nm and about 60 nm, about 10 nm and about 50 nm, about 10 nm and about 40 nm, about 10 nm and about 30 nm, about 10 nm and about 20 nm, about 20 nm and about 100 nm, about 20 nm and about 90 nm, about 20 nm and about 80 nm, about 20 nm and about 70 nm, about 20 nm and about 60 nm, about 20 nm and about 50 nm, about 20 nm and about 40 nm, about 20 nm and about 30 nm, about 30 nm and about 100 nm, about 30 nm and about 90 nm, about 30 nm and about 80 nm, about 30 nm and about 70 nm, about 30 nm and about 60 nm, about 30 nm and about 50 nm, or about 30 nm and about 40 nm in thickness.

A given MPN coating layer may comprise a single metal ion component and a single phenolic component, or may comprise combinations of metal ion and/or phenolic components. In embodiments where 2 or more MPN layers are present, each layer may be identical, or may include different metal ion and/or phenolic components than other layers in the MPN.

In one embodiment, the metal ion component in the MPN comprises one or more cations of aluminium (Al), vanadium (V), chromium (Cr), manganese (Mn), iron (Fe), cobalt (Co), nickel (Ni), copper (Cu), zinc (Zn), zirconium (Zr), molybdenum (Mo), ruthenium (Ru), rhodium (Rh), cadmium (Cd), cerium (Ce), europium (Eu), gadolinium (Gd), terbium (Tb), or combinations thereof. The cations may be in any oxidation state. In various embodiments, an Al cation may be Al³⁺; an Fe cation may be Fe²⁺ and/or Fe³⁺; a Co cation may be Co²⁺ or Co³⁺, etc., as will be understood by those of skill in the art.

In one embodiment, the metal ion component in the MPN comprises an iron cation. In another embodiment, the iron cation comprises Fe³⁺. In various further embodiments, the MPN comprises Cr, Cu, Mn, Mo, and/or Zn cations. In various non-limiting embodiments, the cation comprises one or more of Fe²⁺, Fe³⁺, Cr²⁺, Cr³⁺, Cr⁶⁺, Cu⁺, Cu²⁺, Cu³⁺, Cu⁴⁺, Mn²⁺, Mn³⁺, Mn⁴⁺, Mn⁶⁺, Mn⁷⁺, Mo²⁺, Mo³⁺, Mo⁴⁺, Mo⁶⁺, and/or Zn²⁺. In various non-limiting embodiments, the cation comprises one or more of Fe²⁺, Fe³⁺, Cr³⁺, Cu²⁺, Mn²⁺, Mo²⁺, Mo⁴⁺, Mo⁶⁺, and/or Zn²⁺. In various non-limiting embodiments, the cation comprises one or more of Cr³⁺, Cu²⁺, Mn²⁺, Mo²⁺, and/or Zn²⁺. In various non-limiting embodiments, the cation comprises one or more of Co²⁺, Ni²⁺, Cd²⁺, Al³⁺, V³⁺, Rh³⁺, Ru³⁺, Zr⁴⁺, Eu³⁺, Gd³⁺, and/or Tb³⁺.

Any polyphenol may be incorporated into the MPN coatings as deemed appropriate for an intended use. In some embodiments, the MPNs comprises one or more of flavonoids (including isoflavonoids and neoflavonoids), tannins, condensed tannins, phenolic acids, catechols, lignans, and stilbenes. Other exemplary polyphenols for use in the MPN coatings may be found at https://academic.oup.com/ajcn/article/79/5/727/4690182. In some embodiments, the MPNs comprises one or more of flavonoids, tannins, and phenolic acids. In one embodiment, the polyphenol component of the MPN comprises tannic acid (TA), gallic acid (GA), epigallocatechin gallate (EGCG), or combinations thereof.

In one specific embodiment, the MPNs comprise an iron cation complexed with one or more of tannic acid (TA), gallic acid (GA), and epigallocatechin gallate (EGCG). In a further embodiment, the MPNs comprise Fe³⁺ complexed with one or more of tannic acid (TA), gallic acid (GA), and epigallocatechin gallate (EGCG).

The prokaryotic cell may be a bacterial cell or an archaeal cell, such as a bacterial cell or archaeal cell not pathogenic to an end user, including but not limited to non-pathogenic to humans. Thus, in one embodiment the cell is a bacterial cell. In another embodiment, the cell is an archaeal cell. In a further embodiment, the prokaryotic cell is an anaerobic cell. In various further embodiments, the prokaryotic cell is selected from the group consisting of Bacteroides species including but not limited to Bacteroides thetaiotaomicron; any strain belonging to the genus Lactobacillus including but not limited to L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. casei (Gynophilus), L. coleohominis, Lactobacillus delbrueckii subsp. bulgaricus, L. fornicalis, L. gallinarum, Lactobacillus helveticus, Lactobacillus iners, Lactobacillus jensenii, L. mucosae, and L. vaginalis; any strain belonging to the genus Bifidobacterium including but not limited to B. adolescentis, B. angulation, B. bifidum, B. breve, B. catenulatum, B. infantis, B. lactis, B. longum, B. pseudocatenulatum, Bifidobacterium angulatum, Bifidobacterium animalis subsp. lactis, Bifidobacterium dentium, Bifidobacterium magnum; or any strain belonging to S. thermophiles, Pseudomonas fluorescens, P. protegens, P. brassicacearum, P. aeruginosa; Azospirillum. brabrasilense, A. lipferum, A. halopraeferens, A. irakense; Acetobacter diazotrophicus; Herbaspirillum seropedicae; Bacillus subtilis, Pseudomonas stutzeri, fluorescens, Pseudomonas chlororaphis, P. putida, P. cepacian, P. vesicularis, P. paucimobilis; Bacillus cereus, B. thuringiensis, B. sphaericus; Shewanella oneidensis; Geobacter bemidjiensis, G. metallireducens, G. sulfurreducens, G. uraniireducens, G. lovleyi; Serratia marcescens, Desulfovibrio vulgaris, D. desulfuricans, Dechloromonas aromatic, Deinococcus radiodurans, Methylibium petroleiphilum, Alcanivorax borkumensis, Archaeglobus fulgidus, Haloferax sp., Halobacterium sp., or from the genera of Akkermansia (including but not limited to Akkermansia muciniphila-probiotic), Anaerostipes, Butyricicoccus, Christensenella, Clostridia, Coprococcus, Dorea, Eubacterium, Faecalibacterium, Cutibacterium, such as Cutibacterium acnes, or Roseburia, Staphylococcus, such as Staphylococcus epidermis or Staphylococcus hominis, Weissella viridescens, or the family Coriobacteriaceae; microbial vaccines Bacille Calmette-Guérin (BCG), Ty21a, Lactobacillus, Lactococcus, Streptococcus, Bacillus, Bifidobacterium, and Vitreoscilla, Pseudomonas, Azospirillum, Azotobacter, Klebsiella, Enterobacter, Alcaligenes, Arthrobacter, Burkholderia, Bacillus, and Serratia; cyanobacteria, Anabaena and Nostoc and genera such as Azotobacter, Beijerinckia, and Clostridium; and Rhizobium, and any combinations thereof.

In one specific embodiment, the prokaryotic cell is selected from the group consisting of Bacteroides species including but not limited to Bacteroides thetaiotaomicron.

In one embodiment, the cell is lyophilized. As described herein, the prokaryotic cells of the disclosure can be lyophilized, even in the absence of cannonical cryoprotectants, while retaining viability. The coatings are shown to protects anaerobes from multiple manufacturing and storage stresses, including oxygen exposure and freeze-drying, without requiring additives (e.g. cryoprotectants or ROS scavengers). Further-more, the coating rapidly disassembles under acidic conditions, which is ideal for oral delivery to the gut.

The cells may be modified as deemed appropriate, such as by modifying the MPNs. In one embodiment, the cells further comprise one or more functional groups bound to the MPNs. Any functional group may be employed as suitable for an intended use, including but not limited to bovine serum albumin (BSA) or a targeting biomolecule such as an antibody, an oligonucleotide aptamer, a peptide, or a peptoid, or combinations thereof.

The disclosure further provides compositions comprising a plurality of the prokaryotic cells of any embodiment or combination of embodiments disclosed herein. Prior to this work, MPNs had not successfully been applied to protect prokaryotes. Here, we report the coating of prokaryotic cells, exemplified by E. coli and Bacteroides thetaiotaomicron, with MPNs. Bacteroides species are essential for carbohydrate fermentation in the gut. They can combat dangerous invasive microbes and have the potential to treat colorectal cancer. Thus, the compositions of the disclosure may be used, for example as biotherapeutics and probiotics.

The coated prokaryotic cells in the composition may comprise a plurality of a single coated prokaryotic cell type, or may comprise a plurality of a combination of 2, 3, 4, 5, 6, 7, 8, 9, 10, or more different coated prokaryotic cell types. In one embodiment, the plurality of the coated prokaryotic cells in the composition comprise Bacteroides species including but not limited to Bacteroides thetaiotaomicron. In one embodiment, the cells are lyophilized; in another embodiment, the composition does not include a cryoprotectant.

The prokaryotic cells may be present in the composition in any amount appropriate for an intended use. In one non-limiting embodiment, the prokaryotic cells are present in the composition at between about 10⁴ and 10¹¹ colony forming units (cfu).

The composition may comprise any other suitable components as appropriate for an intended use. In some embodiments, the compositions are for administration to a subject, including but not limited to mammals, such as humans, dogs, cats, horses, cattle, pigs, etc. In one embodiment, the compositions are for administration to a human subject. In these embodiments, the composition may, for example, comprise a probiotic composition. Such probiotic compositions may comprise any other components as suitable for an intended use. In non-limiting embodiments, such additional components may comprise an excipient such as a saccharide, polysaccharide, diluent, lubricant, colorant, binder, coating agent, sweetening agent, anti-caking agent, or suppository base such as triglycerides, polyglycolysed glycerides, polyethylene glycols (PEGs) with oil (lubercant), cocoa butter. Other contemplated excipients are microcrystalline cellulose (as binder/diluent), maltodextrin (as binder/diluent), silicon dioxide (gliding/anti-caking agent), magnesium stearate (as lubricant), hydroxy propyl methyl cellulose (as suspending/viscosity agent). In certain embodiments, the excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, sorbitol, maltitol, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, vitamins, vitamin C, vitamin K, prebiotics, carbohydrates that are not digested by the human body, inulin, fructooligosaccharides, beta-glucan, glucomannon, arabinoxylan oligosaccharides, pectin, short-chain fatty acids, butyrate, acetate, propionate, and combinations thereof.

In some embodiments, the compositions are for cosmetic use. Exemplary prokaryotic cells that may be particularly useful for cosmetic applications include, but are not limited to, Lactobacillus, Lactococcus, Streptococcus, Bacillus, Bifidabacterium, and Vitreoscilla.

In some embodiments, the compositions are for agricultural use. The compositions for agricultural use may include any other components suitable for an intended use, including but not limited to seed coatings, soil, fertilizer, soil additive, a soil stabilizer, soil adjuvant, plant biostimulant, pesticide, and any other components listed above. Exemplary prokaryotic cells that may be particularly useful for agricultural applications include, but are not limited to species such as those that comprise the rhizosphere: Pseudomonas, Azospirillum, Azotobacter, Klebsiella, Enterobacter, Alcaligenes, Arthrobacter, Burkholderia, Bacillus, and Serratia; and those that can perform nitrogen fixation such as cyanobacteria, Anabaena and Nostoc and genera such as Azotobacter, Beijerinckia, and Clostridium; and Rhizobium. The compositions may be formulated in any formulation suitable for an intended use. In one embodiment particularly useful for biotherapeutic/probiotic use, the composition is formulated for oral administration.

In one embodiment, the formulation comprises beads, powders, capsules, tablets, drops, oil suspensions, gels, lozenges, hydrogels, and/or liquid suspensions of the compositions. In further embodiments, the composition may be present in a food or beverage product, including but not limited to milk, yogurt, cheese, cream, chocolate, meat, chewing gum, kombucha or other fermentation products, milk or other beverages, and animal feed or drinking water.

In another aspect, the disclosure provides methods for treating or limiting development of a disorder, comprising administering to a subject in need thereof an amount effective of the non-pathogenic prokaryotic cells and/or compositions of any suitable embodiment herein to treat the disorder. As described herein, the cells retain viability after lyophilization and room temperature storage, greatly facilitating distribution and storage of the living biotherapeutics. In one embodiment, the compositions are administered as a probiotic.

In one embodiment, the non-pathogenic prokaryotic cells and/or compositions are administered orally. As described herein, the MPN coating rapidly disassembles under acidic conditions, which is ideal for oral delivery to the gut.

In a further aspect, the disclosure provides methods for forming a coating of MPNs on prokaryotic cells, comprising adding metal ions and one or more polyphenols to a suspension of prokaryotic cells, and adjusting pH of the suspension to a final pH of between 7.0 and 8.0 by addition of 3-(N-morpholino) propanesulfonic acid (MOPS) buffer. Detailed and non-limiting examples of the methods are provided in the examples that follow. In one embodiment, the method is initiated under nitrogen atmosphere, such as when the prokaryotic cells are anaerobes. The metal ions, polyphenols, prokaryotic cells, and coatings may be any disclosed in detail above. In other embodiments, the method comprises addition of 2, 3, 4, 5, 6, 7, 8, 9, 10, or more MPN layers on the cells.

### EXAMPLE 1

Gut microbes are responsible for processes including synthesis of essential vitamins, cellulose breakdown, and prevention of pathogen invasion. When this community is not in balance, issues ranging from allergies to cancer can result: gastrointestinal infections cause six million deaths each year. With the crucial role that microbes play in human health, there is an urgent need for their development as biotherapeutics to maintain gut homeostasis, treat dis-ease, and preserve or restore barrier integrity.

A key limitation in the production of microbial therapeutics is the need for them to be alive at the time of delivery; currently, most probiotics are produced from a few species based on their ease of production, but advances in our knowledge of the human microbiome have led to demand for new microorganisms as supplements to maintain health or as biotherapeutics to treat disease. Yet, key strains are anaerobic and are nearly impossible to produce with existing technologies.

Manufacture of anaerobes is especially challenging because of both oxygen toxicity and damage during manufacture. Although dry storage is often necessary for administration of microbes, drying processes induce cellular stress and decrease viability. Lyophilization is often used, but as few as 0.1% of cells survive, which is woefully inadequate for therapeutic use. Thus there is no efficient method to protect anaerobes from harsh processing stress and oxygen exposure.

Here, we report a novel self-assembling coating to protect microbes of therapeutic interest from production stresses, including oxygen exposure and lyophilization. This coating is comprised of metal-phenolic networks (MPNs), which are non-covalent coordination complexes of metal ions and natural polyphenols. Unlike coatings that rely on covalent bonds, MPNs adsorb to surfaces through noncovalent inter-actions. Prior to this work, MPNs had yet to be applied to protect prokaryotes. Here, we report the coating of both E. coli and Bacteroides thetaiotaomicron with MPNs. Bacteroides species are essential for carbohydrate fermentation in the gut (3, 24) They can combat dangerous invasive microbes and have the potential to treat colorectal cancer (27-30). Thus, their development as biotherapeutics is of significant interest, but as anaerobes, their production remains a challenge. We have developed a generalizable protocol to coat microbes with MPNs, characterized the coatings, and demonstrated that MPNs protect next-generation biotherapeutics during lyophilization, even in the absence of canonical cryoprotectants.

### Results and Discussion for Example 1

### Coating Microbes With MPNs

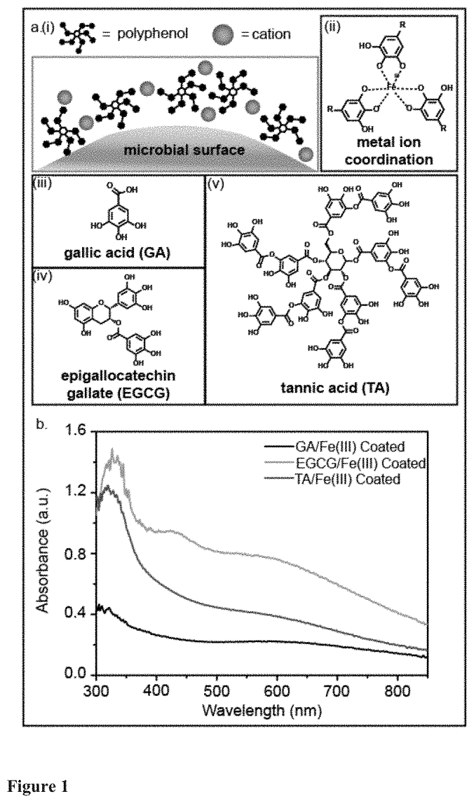

We have successfully generated MPNs on microbes using Fe³⁺ ions and one of three polyphenols: tannic acid (TA), gallic acid (GA), or epigallocatechin gallate (EGCG) (FIG. 1a-b). The MPN shell formed consistently on microbial suspensions following the addition of the MPN components and pH adjustment to 7.0-8.0 both in air and under N₂ atmosphere. The formation of a stable MPN complex was confirmed by the appearance of a ligand-to-metal charge transfer (LMCT) band between 500-600 nm in UV-visible spectra (FIG. 1c, 5).

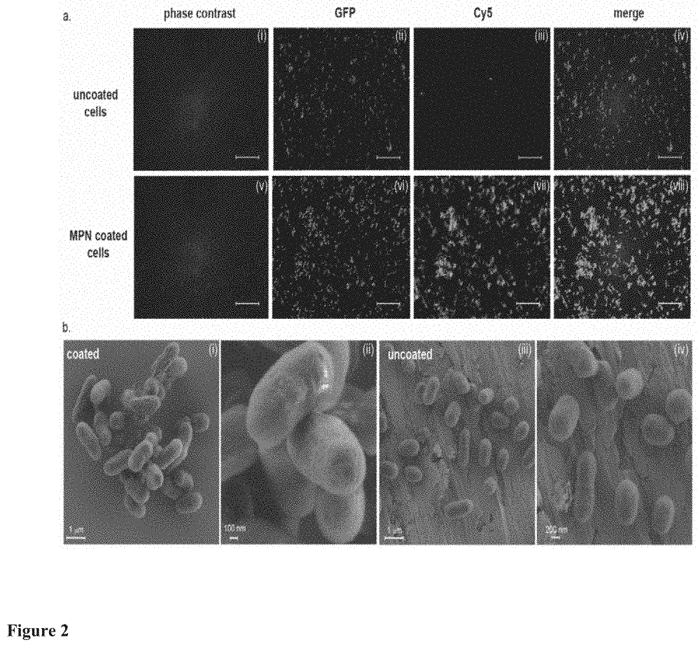

To verify that the MPN shell forms on the surface of microbes, a fluorophore-conjugated protein (BSA-Alexa Fluor 647) was incubated with MPN-encapsulated E. coli that express green fluorescent protein (GFP). The BSA adheres preferentially to the MPN, as can be seen by fluorescence microscopy (FIG. 2a). The core-shell structure confirms the formation of a contiguous, homogeneous shell on individual microbes (FIG. 2a). Scanning electron microscopy (SEM) images further confirmed MPN assembly, showing individual E. coli encapsulated in TA-Fe³⁺ complexes, as indicated by their rough surface (FIG. 2b). Uncoated E. coli were also found to have shorter lengths and widths (1.25±0.29 μm and 0.642±0.044 μm, respectively) than coated cells (1.49±0.25 μm and 0.710±0.060 μm, respectively), which can be explained by MPN protection of the coated microbes under vacuum. When taken together, these results confirm the successful formation of MPNs on individual microbes with maintenance of native cell morphology.

### Viability of Microbes Following Coating

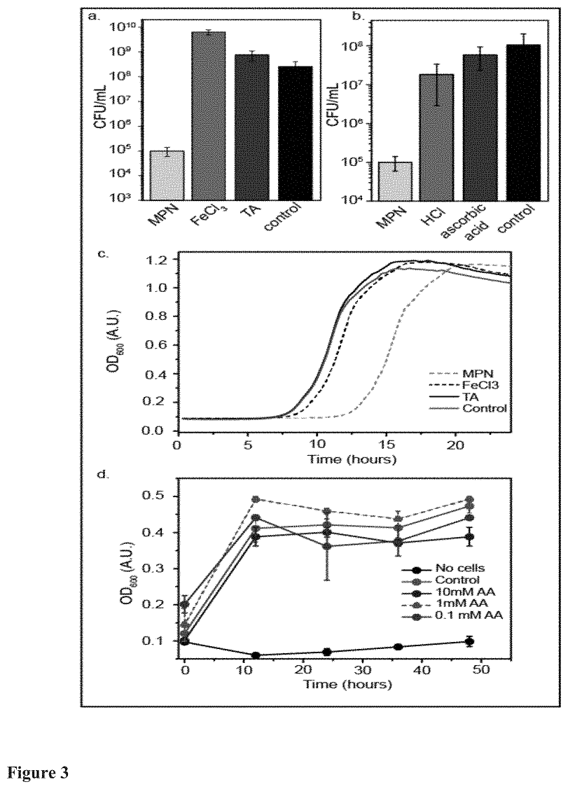

For MPN use as a protective agent, cells must remain viable after coating. We therefore monitored bacterial growth by OD600 and colony forming unit (CFU) measurements. Serial dilutions of encapsulated E. coli were placed on lysogeny broth (LB) agar plates or used to inoculate LB medium, with uncoated cells as controls. After 48 h at 37° C., colonies were counted on plates. E. coli OD600 was monitored continuously using a plate reader. B. thetaiotaomicron OD600 was evaluated either by a plate reader under N₂ atmosphere or at defined timepoints in air. The log (CFU/mL) was 8.41±0.58 for native E. coli and 5.00±0.38 for MPN-encapsulated E. coli, demonstrating that coated cells divide three orders of magnitude less in a given time as compared to uncoated controls (FIG. 3a). Interestingly, for cells treated with only FeCl3 or TA, the log(CFU/mL) was 9.80±0.21 and 8.88±0.40, respectively, slightly higher for coated than uncoated cells, likely due to the addition of soluble iron, which is a micronutrient and is involved in bacterial growth. These results indicate that the MPN shell inhibits cell division in both solid and liquid culture. Based on prior work with eukaryotes, these results are unsurprising, as MPNs have been shown to serve as a rigid barrier that prevents cells from contacting nutrients in solid culture.

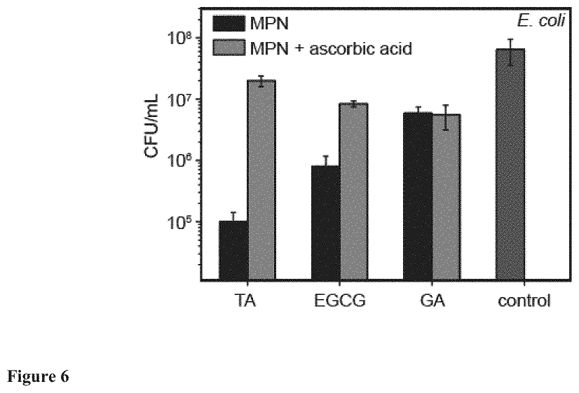

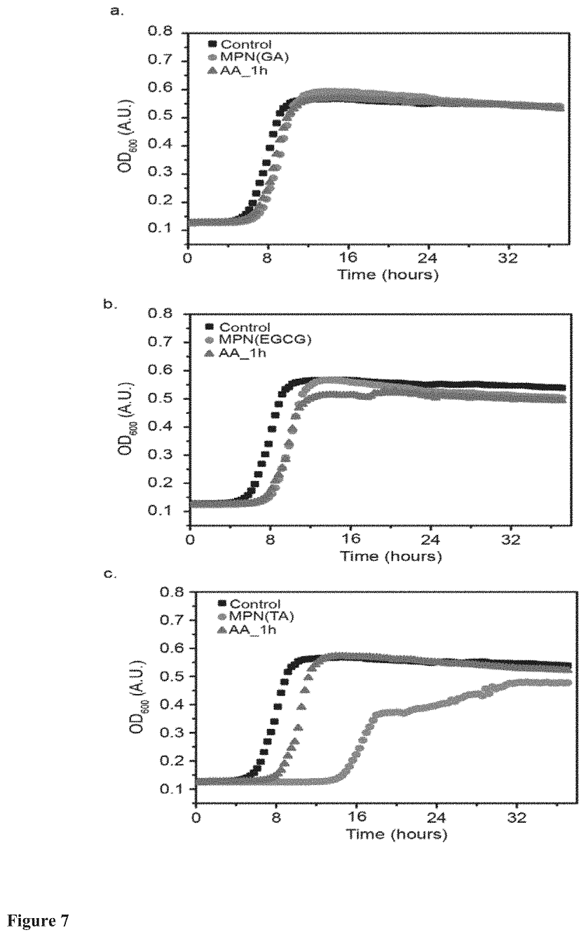

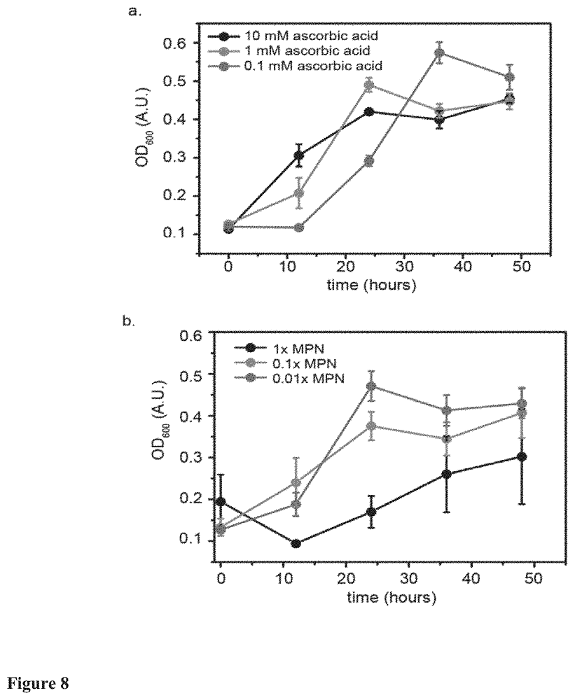

To further explore our findings, we evaluated whether cell division inhibition was equivalent across MPNs formed with different phenols (TA, GA, and EGCG). In short, we found that it was not. The EGCG MPNs showed a 100-fold decrease in CFU/mL and GA MPNs a 10-fold decrease as compared to uncoated cells (FIG. 6-7), indicating that structure of the phenol is important to the physical proper-ties of the coatings. A similar trend was observed for growth of MPN-encapsulated E. coli in liquid medium, with exponential growth delayed to 12.5 h after encapsulation (FIG. 3c). Based on our hypothesis that the rigidity impacts the delay in cell division, we would similarly expect the thickness of the coating to influence this delay. As expected, under anaerobic conditions, the exponential growth delay of MPN-encapsulated B. thetaiotaomicron increased as the thickness of the shell increased (FIG. 8b).

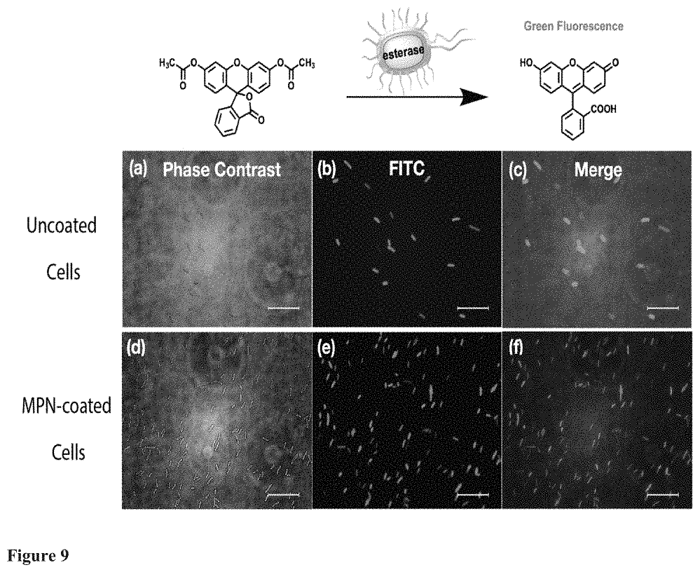

For microbial biotherapeutics, cells must be alive at the time of delivery, making division inhibition a potential concern. However, as cell division inhibition does not indicate inviability, we performed a fluorescein diacetate assay (FDA) following encapsulation to evaluate cellular activity. FDA is a substrate for esterases, enabling measurement of enzymatic activity and membrane integrity. We observed significant fluorescence from encapsulated cells (FIG. 9), confirming that the coated cells are viable and metabolically active. Although our coatings do not affect viability, their impact on growth could pose a challenge for bio-therapeutic delivery if cells are unable to divide. We therefore investigated MPN disassembly.

### Coating Disassembly

MPNs are pH sensitive and readily degraded upon treatment with a mild acid (conditions similar to stomach acid), enabling their “on demand” dissolution. Immediately following the addition of acid, the color of the cell suspension changed to white, indicating removal of the MPN shell. After one hour of incubation in 10 mM HCL, the log(CFU/mL) for coated E. coli increased to 7.26±1.07, and the delay in exponential growth (teg) decreased to eight hours, similar to that of uncoated cells (FIG. 3b). Similarly, the teg for Fe³⁺-TA encapsulated B. thetaiotaomicron decreased to less than one hour following only five min of treatment with 10 mM L-ascorbic acid (FIG. 8a). With lower concentrations of L-ascorbic acid, slower recovery of teg was observed (FIG. 8a), and no growth rate recovery was observed with 0.1 mM acid. Importantly, the growth rate of the uncoated B. thetaiotaomicron is not impacted by this acid treatment (FIG. 3d). These results indicate that the MPN shell is degraded under conditions relevant to oral delivery, as the coated product would pass through the stomach (with exposure to stomach acid) prior to entering the gut. Thus, this on-demand disassembly is compatible with probiotics and biotherapeutics that require both long-term preservation and controlled release in situ.

### Lyophilization Protection

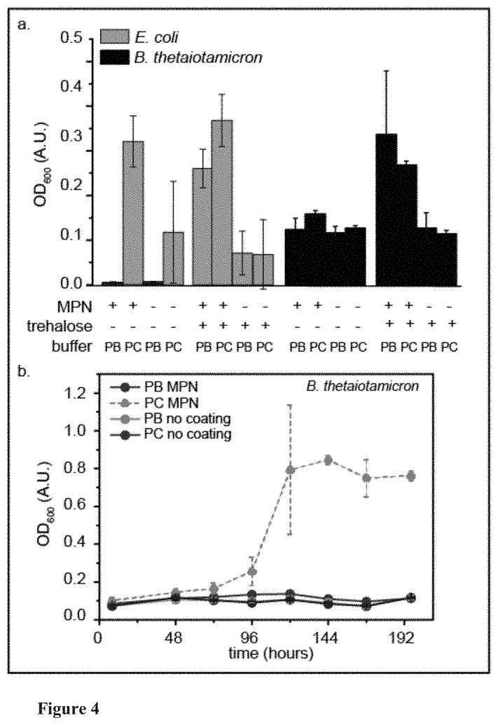

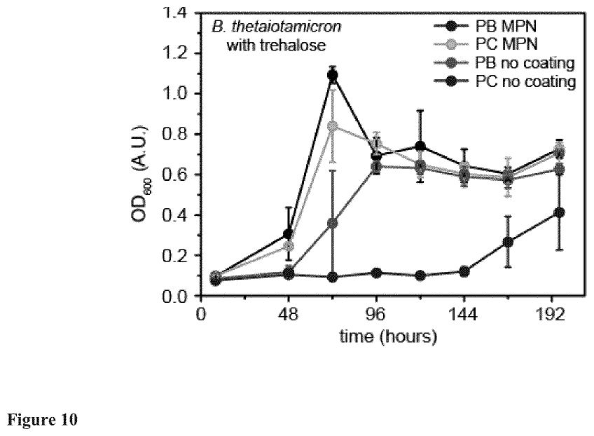

Finally, we determined if MPN coatings provide protection during a key processing step: lyophilization. Despite its extensive use to stabilize therapeutics, for some microbes, less than 0.1% survive, even with common cryoprotect-ants. Because MPNs are structurally rigid, we hypothesized that they would provide additional protection during lyophilization. Coated cells were flash-frozen and lyophilized in different buffers. OD600 was monitored following reconstitution in the proper liquid culture medium. Impressively, both MPN-coated E. coli and B. thetaiotaomicron demonstrate higher OD600's after a 48 h incubation as compared to the uncoated controls in both phosphate (PB) and phosphate citrate (PC) buffer supplemented with trehalose (FIG. 4a; FIG. 10).

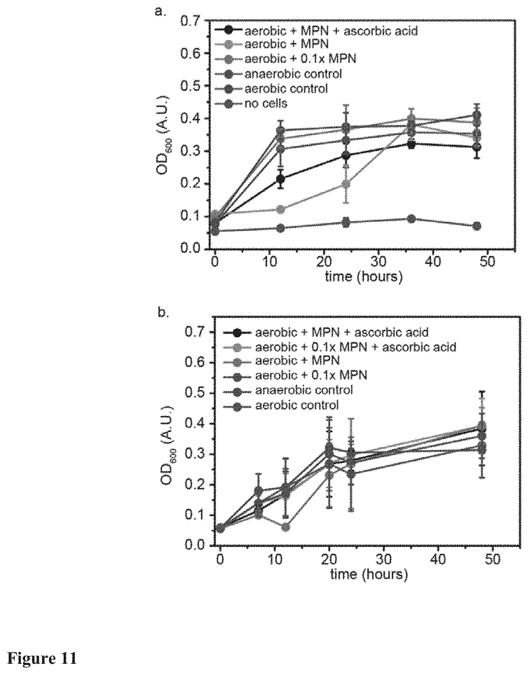

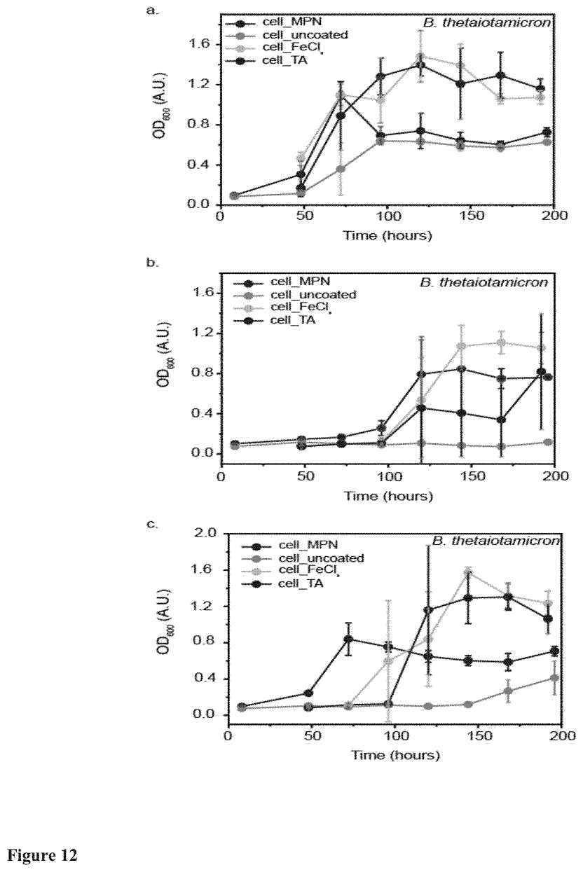

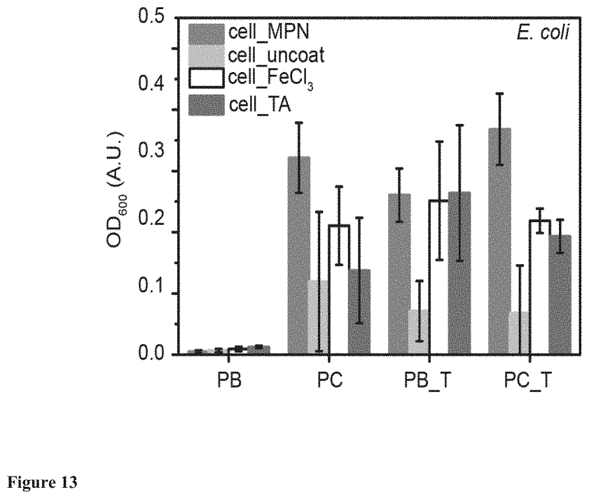

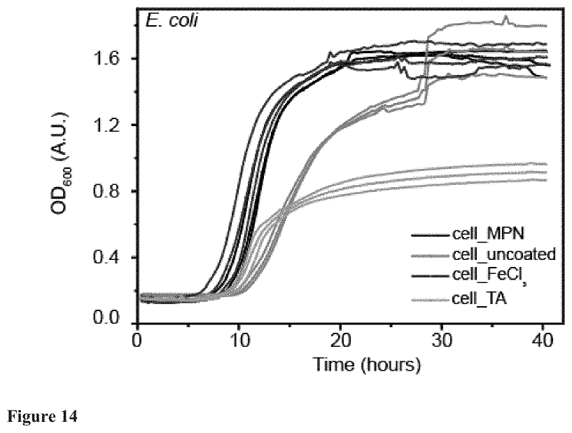

Of note, for coated E. coli in cryoprotectant-free PC buffer, a three-fold increase in OD600 was observed after 48 h (FIG. 4a). Remarkably, in similar conditions, B. thetaiotaomicron reached exponential phase after 96 h of incubation with no growth observed for the uncoated control (FIG. 4b). These improvements indicate that MPN-coated microbes have a higher survival rate after lyophilization even in the absence of conventional cryoprotectants. Furthermore, for anaerobes, Fe³⁺-TA likely provides an additional layer of protection against ROS, as is evidenced by the fact that B. thetaiotaomicron showed faster recovery following oxygen exposure and both strains lyophilized in the presence of FeCl3 showed a higher OD600's as compared to uncoated controls (FIG. 11-14). The MPN-coated cells always demonstrate the fastest growth rate after reconstitution, which highlights MPN protective ability.

In summary, we have developed a self-assembled cellular coating for microbes to maintain viability during processing. This simple coating is comprised of natural phenols and iron chloride, which are generally regarded as safe by the Food and Drug Administration. We demonstrate that this biocompatible encapsulation protects anaerobes from multiple manufacturing and storage stresses, including oxygen exposure and freeze-drying, without requiring additives (e.g. cryoprotectants or ROS scavengers). Furthermore, the coating rapidly disassembles under acidic conditions, which is ideal for oral delivery to the gut. Thus, we have tackled key issues with the development of microbes as living biotherapeutics. This coating strategy is extendable to protect nearly any cellular biotherapeutic.

### REFERENCES FOR EXAMPLE 1

(1) Rowland, I.; Gibson, G.; Heinken, A.; Scott, K.; Swann, J.; Thiele, I.; Tuohy, K. Gut Microbiota Functions: Metabolism of Nutrients and Other Food Components. Eur. J. Nutr. 2018, 57 (1), 1-24.
(2) Schwabe, R. F.; Jobin, C. The Microbiome and Cancer. Nat. Rev. Cancer 2013, 13 (11), 800-812.
(3) Wexler, H. M. Bacteroides: The Good, the Bad, and the Nitty-Gritty. Clin. Microbiol. Rev. 2007, 20 (4), 593-621.
(4) Jagai, J. S.; Smith, G. S.; Schmid, J. E.; Wade, T. J. Trends in Gastroenteritis-Associated Mortality in the United States, 1985-2005: Variations by ICD-9 and ICD-10 Codes. BMC Gastroenterol. 2014, 14, 211.
(5) Wu, H. J.; Wu, E. The Role of Gut Microbiota in Immune Homeostasis and Autoimmunity. Gut Microbes 2012, 3 (1).
(6) Fijan, S. Microorganisms with Claimed Probiotic Properties: An Overview of Recent Literature. Int. J. Environ. Res. Public Health 2014, 11 (5), 4745-4767.
(7) O'Toole, P. W.; Marchesi, J. R.; Hill, C. Next-Generation Probiotics: The Spectrum from Probiotics to Live Biotherapeutics. Nature Microbiology. Nature Publishing Group April 2017, pp 1-6.
(8) El Hage, R.; Hernandez-Sanabria, E.; Van de Wiele, T. Emerging Trends in “Smart Probiotics”: Functional Consideration for the Development of Novel Health and Industrial Applications. Frontiers in Microbiology. Frontiers Media S.A. September 2017, p 1889.
(9) Olle, B. Medicines from Microbiota. Nat. Biotechnol. 2013, 31 (4), 309-315.
(10) Terpou, A.; Papadaki, A.; Lappa, I. K.; Kachrimanidou, V.; Bosnea, L. A.; Kopsahelis, N. Nutrients-11-01591 (1). Pdf. Nutrients 2019, 11 (7), 32.
(11) Murphy, M. P.; Holmgren, A.; Larsson, N.-G.; Halliwell, B.; Chang, C. J.; Kalyanaraman, B.; Rhee, S. G.; Thornalley, P. J.; Partridge, L.; Gems, D.; et al. Unraveling the Biological Roles of Reactive Oxygen Species. Cell Metab. 2011, 13 (4), 361-366.
(12) Wang, Y.; Hekimi, S. Mitochondrial Dysfunction and Longevity in Animals: Untangling the Knot. Science 2015, 350 (6265), 1204-1207.
(13) Hayyan, M.; Hashim, M. A.; AlNashef, I. M. Superoxide Ion: Generation and Chemical Implications. Chem. Rev. 2016, 116 (5), 3029-3085.
(14) França, M. B.; Panek, A. D.; Eleutherio, E. C. A. Oxidative Stress and Its Effects during Dehydration. Comparative Biochemistry and Physiology—A Molecular and Integrative Physiology. Elsevier Inc. April 2007, pp 621-631.
(15) Broeckx, G.; Vandenheuvel, D.; Claes, I. J. J.; Lebeer, S.; Kiekens, F. Drying Techniques of Probiotic Bacteria as an Important Step towards the Development of Novel Pharmabiotics. International Journal of Pharmaceutics. Elsevier B.V. May 2016, pp 303-318.
(16) Miyamoto-Shinohara, Y.; Imaizumi, T.; Sukenobe, J.; Murakami, Y.; Kawamura, S.; Komatsu, Y. Survival Rate of Microbes after Freeze-Drying and Long-Term Storage. Cryobiology 2000, 41 (3), 251-255.
(17) Minelli, E. B.; Benini, A. Relationship between Number of Bacteria and Their Probiotic Effects. Microb. Ecol. Health Dis. 2008, 20 (4), 180-183.
(18) Leslie, S. B.; Israeli, E.; Lighthart, B.; Crowe, J. H.; Crowe, L. M. Trehalose and Sucrose Protect Both Membranes and Proteins in Intact Bacteria during Drying. Appl. Environ. Microbiol. 1995, 61 (10), 3592-3597
(19) Crowe, L. M.; Womersley, C.; Crowe, J. H.; Reid, D.; Appel, L.; Rudolph, A. Prevention of Fusion and Leakage in Freeze-Dried Liposomes by Carbohydrates. BBA—Biomembr. 1986, 861 (C), 131-140.
(20) Morgan, C. A.; Herman, N.; White, P. A.; Vesey, G. Preservation of Micro-Organisms by Drying; A Review. Journal of Microbiological Methods. Elsevier August 2006, pp 183-193.
(21) Ejima, H.; Richardson, J. J.; Caruso, F. Metal-Phenolic Networks as a Versatile Platform to Engineer Nanomaterials and Biointerfaces. Nano Today 2017, 12, 136-148.
(22) Park, J. H.; Kim, K.; Lee, J.; Choi, J. Y.; Hong, D.; Yang, S. H.; Caruso, F.; Lee, Y.; Choi, I. S. A Cytoprotective and Degradable Metal-Polyphenol Nanoshell for Single-Cell Encapsulation. Angew. Chemie Int. Ed. 2014, 11, 12420-12425.
(23) Guo, J.; Suma, T.; Richardson, J. J.; Ejima, H. Modular Assembly of Biomaterials Using Polyphenols as Building Blocks. ACS Biomater. Sci. Eng. 2019, 5 (11), 5578-5596.
(24) Flint, H. J.; Scott, K. P.; Duncan, S. H.; Louis, P.; Forano, E. Microbial Degradation of Complex Carbohydrates in the Gut. Gut Microbes 2012, 3 (4).
(25) Charbonneau, M. R.; Isabella, V. M.; Li, N.; Kurtz, C. B. Developing a New Class of Engineered Live Bacterial Therapeutics to Treat Human Diseases. Nat. Commun. 2020, 11 (1), 1738.
(26) Fan, H.; Chen, Z.; Lin, R.; Liu, Y.; Wu, X.; Puthiyakunnon, S.; Wang, Y.; Zhu, B.; Zhang, Q.; Bai, Y.; et al. Bacteroides fragilis Strain ZY-312 Defense against Cronobacter sakazakii-Induced Necrotizing Enterocolitis In Vitro and in a Neonatal Rat Model. mSystems 2019, 4 (4).
(27) Lee, Y. K.; Mehrabian, P.; Boyajian, S.; Wu, W.-L.; Selicha, J.; Vonderfecht, S.; Mazmanian, S. K. The Protective Role of &lt;Em&gt;Bacteroides Fragilis&lt;/Em&gt; in a Murine Model of Colitis-Associated Colorectal Cancer. mSphere 2018, 3 (6), e00587-18.
(28) Sittipo, P.; Lobionda, S.; Choi, K.; Sari, I. N.; Kwon, H. Y.; Lee, Y. K. Toll-Like Receptor 2-Mediated Suppression of Colorectal Cancer Pathogenesis by Polysaccharide A From Bacteroides fragilis. Frontiers in Microbiology. 2018, p 1588.
(29) Xie, W.; Guo, Z.; Zhao, L.; Wei, Y. Metal-Phenolic Networks: Facile Assembled Complexes for Cancer Theranostics. Theranostics 2021, 11 (13), 6407-6426.
(30) Zhong, Q. Z.; Pan, S.; Rahim, M. A.; Yun, G.; Li, J.; Ju, Y.; Lin, Z.; Han, Y.; Ma, Y.; Richardson, J. J.; et al. Spray Assembly of Metal-Phenolic Networks: Formation, Growth, and Applications. ACS Appl. Mater. Interfaces 2018, 10 (39), 33721-33729.
(31) Cross, J. H.; Bradbury, R. S.; Fulford, A. J.; Jallow, A. T.; Wegmüller, R.; Prentice, A. M.; Cerami, C. Oral Iron Acutely Elevates Bacterial Growth in Human Serum. Sci. Rep. 2015, 5, 1-7.
(32) Yun, G.; Richardson, J. J.; Biviano, M.; Caruso, F. Tuning the Mechanical Behavior of Metal-Phenolic Networks through Building Block Composition. ACS Appl. Mater. Interfaces 2019, 11 (6), 6404-6410.
(33) Shin, J. M.; Choi, G. H.; Song, S. H.; Ko, H.; Lee, E. S.; Lee, J. A.; Yoo, P. J.; Park, J. H. Metal-Phenolic Network-Coated Hyaluronic Acid Nanoparticles for Ph-Responsive Drug Delivery. Pharmaceutics 2019, 11 (12), 1-11.

### Materials and Methods For Example 1

Chemicals and Reagents. All chemicals were reagent grade and used without further purification unless otherwise stated. Iron (III) chloride (FeCl₃, 97%), tannic acid, (−)-epigallocatechin gallate (EGCG, 80%), gallic acid monohydrate (98%), L-ascorbic acid (99%), 3-(N-morpholino) propanesulfonic acid (MOPS, 99.5%), sodium hydroxide (98%), hydrochloride acid (37%), fluorescein diacetate, acetone (99.5%), D-(+)-trehalose dihydrate (99%), sodium phosphate dibasic (99%), sodium phosphate monobasic (99%), citric acid (99.5%) and glutaraldehyde solutions (25% in H₂O) were purchased from Sigma-Aldrich. Sodium chloride, absolute ethanol (molecular biology grade), yeast extract (granulated), and agar (granulated) were purchased from Fisher BioReagents. Tryptone (bacteriological grade) and yeast extract (bacteriological grade) were purchased from Apex Bioresearch Products. Green tea extract (50% EGCG) was purchased from Bulk Supplements. Ultrapure water was generated from an ELGA PURELAB™ Quest UV (Model number: PQDIUVM1NSP). Alexa Fluor® 647 conjugated albumin from bovine serum (BSA-Alexa Fluor® 647) was purchased from Life Technologies. Lysogeny broth (LB) broth liquid media were prepared with 10 g of tryptone, 5 g of yeast extract and 10 g of sodium chloride in 1 L of nanopure water (adjust pH to 7.0 with sodium hydroxide) and used after autoclaving (20 min, 121° C.). LB agar plates were prepared on Petri dishes with 20 mL of LB agar solution (10 g of tryptone, 5 g of yeast extract, 10 g of sodium chloride and 15 g of agar in 1 L of nanopure water). BRU broth and yeast casitone fatty acids agar plates with carbohydrates (YCFAC) were purchased from Anaerobe Systems and transferred into the glovebox before use.

Analysis and Measurement. Scanning electron microscopy (SEM) imaging was performed with a Zeiss Merlin high-resolution scanning electron microscope (Jena, Germany) with an accelerating voltage of 1 kV (with a resolution of 1.4 nm). Optical density or absorbance was measured with a Biotek synergy mx microplate reader (BioTek Instruments, USA), Byonoy Absorbance 96 plate reader (Byonoy, Germany), or NanoDrop™ One Microvolume UV-Vis Spectrophotometer (Thermo Scientific, USA). The cells were observed with a 710 confocal laser-scanning microscope (CLSM) (Zeiss, Germany) or Revolve Fluorescence Microscope (Echo, USA).

Bacterial Strains and Culture. Strains (Escherichia coli MG1655, DH10β, 25922GFP; Bacteroides thetaiotaomicron VPI-5482 wildtype (ATCC 29148)) were used in this study. Anaerobic culture was performed with an incubator located inside a VAC Genesis Glovebox containing a humidified atmosphere of nitrogen (oxygen level remain below 0.3 ppm by oxygen purifier throughout the experiment). The E. coli strains were maintained on LB agar plates that were supplemented with 50 μm/mL kanamycin as necessary. During routine propagation, B. thetaiotaomicron strains were maintained on YCFAC agar plates (Anaerobe System) in the glovebox. For growth assays and surface coating, B. thetaiotaomicron and E. coli strains were grown anaerobically in LB or BRU broth respectively.

Cells were prepared for metal phenolic network (MPN) surface coating as follows: E. coli strains stored in 20% glycerol at −80° C. were freshly streaked onto LB agar plates and aerobically incubated for ˜16 h at 37° C. B. thetaiotaomicron strains stored in 20% glycerol at −80° C. were freshly streaked onto YCFAC agar plates in the glovebox and anaerobically incubated for ˜40 h at 37° C. Single colonies were used to inoculate LB or BRU broth. Inoculated cultures were then incubated for ˜16 h (E. coli) or ˜40 h (B. thetaiotaomicron) at 37° C. Stationary-phase cultures were washed by transferring cells to centrifuge tubes (For B. thetaiotaomicron, inside the anaerobic chamber) and spinning at 6000×g for 20 min. Supernatant was exchanged with nanopure water or buffer as necessary. Two washes were performed. Following the final wash, cells were concentrated to a 4× stock (OD₆₀₀ of 4.0) and were used immediately following concentration.

Metal Phenolic Network (MPN) Encapsulation. A single colony of E. coli was selected from an LB agar plate and cultured in the LB broth liquid media with continuous shaking at 37° C. for ˜16 h. The cell suspensions were washed with naopure water three times and dispersed in nanopure water. In a typical cell encapsulation process, the 125 μL aqueous solution of tannic acid (1.6 mg mL⁻¹) and the 125 μL aqueous solution of FeCl₃ (0.24 mg mL⁻¹) were added sequentially to the aqueous suspension of cells (250 μL OD₆₀₀ of 4.0). The resulting suspension was mixed vigorously for 10 s, and 0.5 mL of MOPS buffer (20 mM, pH 7.4) were added to the suspension for the formation of a stable MPN shell. The resulting encapsulated cells were washed with DI water three times to remove any residual starting materials.^{[11]} The coating process from addition of tannic acid and FeCl₃ to washing with nanopure water was repeated two times as necessary. The concentrations of the tannic acid or FeCl₃ were adjusted as necessary. For other phenols, equivalent amounts of gallic acid (1.5 mg mL⁻¹) or epigallocatechin gallate (2.7 mg mL⁻¹, 50% from tea extract) and an equal volume of aqueous solution of FeCl₃ (0.24 mg mL⁻¹) were applied for encapsulation. For cell division evaluation, 5 mM HCl or 0.1 mM-10 mM L-ascorbic acid solution was added to the encapsulated cells for either 5 min or 1 hour.

SEM Analysis of Bacterial Cells. Bacterial cells either coated in MPNs or uncoated were fixed in a 2% glutaraldehyde solution for 2 hours. The cell suspension was rinsed with DI water or fresh buffer and pelleted by centrifugation (6000×g, 20 min, 3 times). Aqueous buffer solution was slowly exchanged with ethanol using a series of ethanol dilutions (35%, 50%, 70%, 80%, 95% and 100%), with pelleting between each step (6000×g, 20 min). The ethanol solution containing concentrated cell suspension was drop-cast onto a silicon wafer chip, dried in a critical point dryer, and sputter-coated with platinum as necessary prior to SEM analysis. SEM images of the E. coli uncoated and coated with MPNs of Fe³⁺ and TA were analyzed using Fiji ImageJ software. This software was used to quantify the length and diameter of the rod-shaped bacteria. The scale was set for each individual image; for images with scale of 1 μm, the measured scale bar was equivalent to 90 pixels and for images with scale of 200 nm, the scale bar was equivalent to 35 pixels. After setting the scale for each image, a total of 120 measurements, including length and diameter, were registered for both, the coated and uncoated E. coli images. The measurements were specifically registered for E. coli that could be accurately measured from the angle at which the SEM image was captured. The resulting length for the uncoated E. coli was 1.25±0.29 μm and for the MPN coated E. coli it increased to 1.49±0.25 μm. The resulting diameter for the uncoated E. coli was 0.642±0.044 μm and for the MPN coated E. coli it increased to 0.710±0.060 μm.

Confocal Laser-Scanning Microscope (CLSM). Confocal fluorescence images were recorded on a Zeiss CLSM 710. Excitation was performed with an Ar laser at a wavelength of 405, 488 or 633 nm, and the emission was monitored at 493-556 nm or 650-680 nm. For viability evaluation, a fluorescein diacetate stock solution (5 mg mL⁻¹) was prepared in acetone, and 4 μL of the stock solution was added into the 500 μL of cell suspension (OD=4). After 20 min, the cells were washed with DI water and then observed under CLSM. The encapsulated cells were then incubated with an aqueous solution of BSA-Alexa 647 (0.4 mg mL⁻¹) for 15 min to selectively label MPNs. After that, the cell suspension was washed with nanopure water three times before CLSM. Before CLSM observation, an aqueous solution of gelatine (1.0 mg mL⁻¹) was added into the cell suspension at 40° C. and cooled to ambient temperature. The resultant viscous mixture was dropcast onto untreated glass substrates for observation. Preparation of Lyophilized Cells. E. coli were incubated for −16 h in 2 mL LB at 37° C. from 20% frozen glycerol stocks (stored at −80° C.). The cells were then pelleted by centrifugation (6000×g, 20 min, 3 times) and washed with DI water. After MPN encapsulation, cells were concentrated in buffer with 100 mM trehalose as a cryoprotectant to a final OD₆₀₀ of 2.0 or 5.0, depending upon the use. The buffer for resuspension was phosphate citrate buffer (PC) or phosphate buffered saline (PB) for subsequent growth evaluation. 1 mL aliquots of the cell culture at specific OD₆₀₀ values were flash frozen in liquid N₂, lyophilized under vacuum and stored at −20° C. until further use. Before use, the lyophilized cells were reconstituted in sterile water or LB broth as necessary. The preparation of lyophilized B. thetaiotaomicron was carried out in a similar manner in the glovebox.

Bacterial Growth/Viability Assessment. Growth assays were performed in sterile 96-well plates, and OD₆₀₀ was monitored by plate reader. Bacterial viability was determined by colony forming units (CFU) counting. For CFU counting, serial dilutions of E. coli suspensions before and after MPN encapsulation were plated on LB agar, and incubated aerobically for 18 hours. The growth assessment of B. thetaiotaomicron was carried out in glovebox. For growth curve evaluation following oxygen exposure, the encapsulated and uncoated B. thetaiotaomicron suspensions in sterile centrifuge tubes were removed from glovebox and agitated using sterile pipette tips periodically. After 1 h, the cell suspensions were transferred into the glovebox and diluted to initiate growth assay evaluation.
[1] J. H. Park, K. Kim, J. Lee, J. Y. Choi, D. Hong, S. H. Yang, F. Caruso, Y. Lee, I. S. Choi, Angew. Chemie—Int. Ed. 2014, 53, 12420-12425.

EXAMPLE 2

### Abstract

Bacillus subtilis are important probiotic microbes currently formulated for delivery as spores, but their ability to germinate in the gut remains debatable. To optimize their application, cells should be delivered in their vegetative state, but the sensitivity of B. subtilis prevents this. This example demonstrates that through the application of self-assembled MPN cellular coatings, B. subtilis are protected from lyophilization stresses. Delivery to the gut involves exposure to acidic conditions in the form of stomach acid and intestinal fluid. MPN coatings rapidly disassemble upon mild acid treatment but were found to protect B. subtilis from the negative impacts of the acid. Overall, MPNs were found to protect vegetative B. subtilis cells from lyophilization stress.

### Introduction

Bacillus subtilis are gram-positive and spore forming, enabling their production and delivery through sporulation. The spores of these microbes are highly tolerant to environmental stressors, but spores are dormant. Thus, there has been significant debate over the efficacy of spore-based probiotics including B. subtilis because germination must occur in the gastrointestinal (GI) tract for most host benefits. Thus, we sought to employ MPN coatings as “synthetic spore coats” on non-sporulated cells to enable their rapid recovery in the GI tract. The importance of a healthy gut microbiome and the negative health effects of an unbalanced microbiome are well known, resulting in an urgent need to improve probiotics for more effective gut implantation

We evaluated the impact of varying the polyphenol and the metal ion on the cellular protection effects of MPNs to protect Bacillus subtilis from freeze-drying process. We demonstrate that MPNs are readily self-assembled on silica bead substrates using different metal ions (Fe³⁺, Mn²⁺, Zn²⁺, and Al³⁺) and phenols (TA, EGCG and GA) for spectroscopic characterization. The coating process was found to be biocompatible and showed negligible adverse effects on microbial viability. Furthermore, Fe³⁺-TA- and Fe³⁺-GA-coated B. subtilis in cryoprotectant-free buffer showed faster recovery following lyophilization. Remarkably, after simple L-ascorbic acid treatment (which mimics the pH of the GI tract), MPN-coated B. subtilis reached exponential phase after incubation for 16 h, whereas no growth was observed for the uncoated control. Overall, our results demonstrate that MPN coatings protect different microbes from lyophilization stresses, and the optimal components of these coatings may differ based on the strain of interest. Specifically, we have shown the protection of non-sporulated B. subtilis during processing with MPN components with the ready subsequent removal of the coating in a GI environment.

### Results and Discussion

### Spectroscopic Characterization of MPN Assemblies

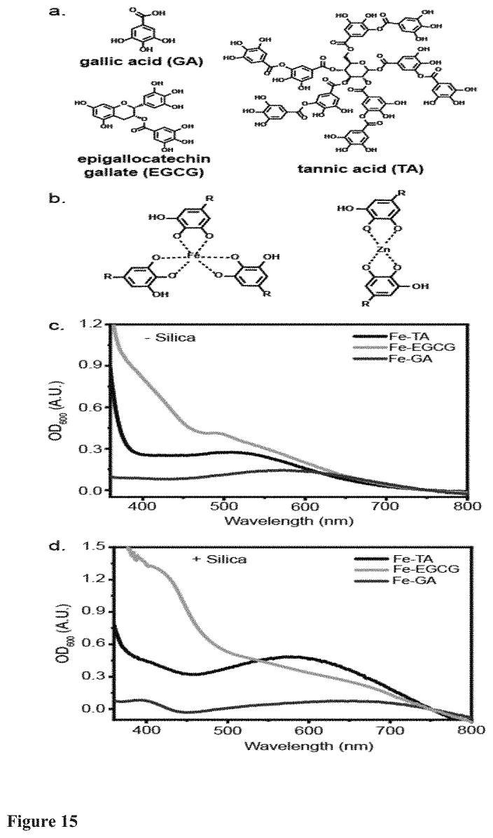

By simply mixing a series of metal ions and polyphenols, we could gain access to a library of MPN coatings with tunable mechanical properties. Biocompatible and biorelevant metal ions (Fe³⁺, Mn²⁺, Zn²⁺, and Al³⁺) and phenols (TA, EGCG, and GA) were selected for this evaluation (FIG. 15). In addition to the structure of the phenols and the valency of the metal ions, the pH of the coordination solution is also important for MPN formation, as alkaline conditions are necessary for effective chelation between the metal ion and hydroxyl groups. To evaluate MPN formation using various metals and polyphenols, solutions containing metal ions and polyphenols were mixed. An equal volume of MOPS buffer (20 mM, pH 7.5) was added to the pre-complexed MPN solution, with a final MOPS concentration of 10 mM when in solution with the MPNs. Interestingly, upon addition of MOPS buffer, a weak ligand-to-metal charge transfer (LMCT) band between 400 nm to 700 nm was observed in the UV-Vis spectrum, indicating the formation of the MPN complex.

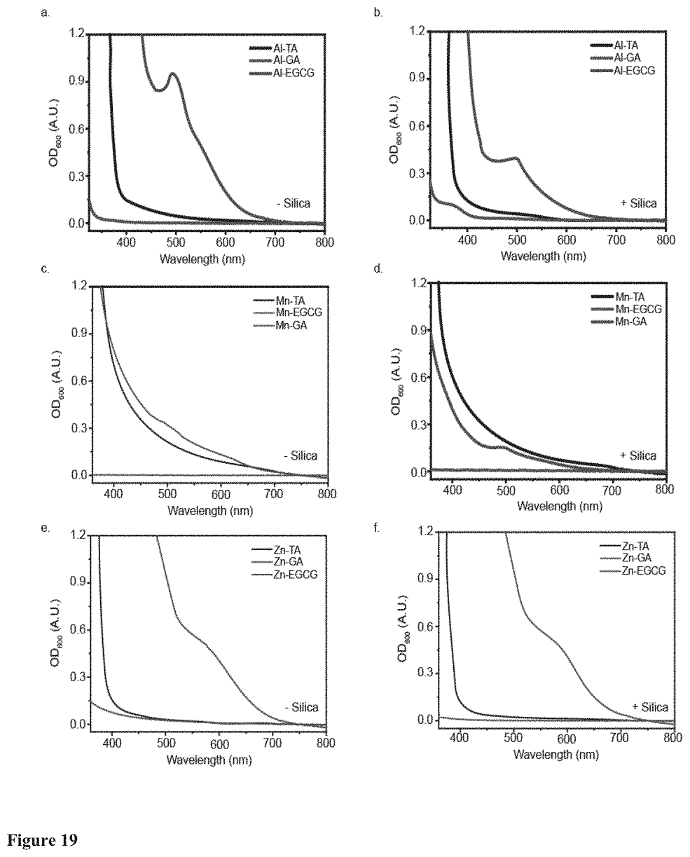

To evaluate the impact of micron-scale substrates for MPN formation, we evaluated the encapsulation of negative-charged, micron-scale silica particles. Excitingly, upon addition of silica to the MPN solution, followed by pH adjustment, stronger absorbance bands were shown, indicating the deposition of MPNs on silica particles (FIG. 15). Some metal-phenol pairs (Fe³⁺-TA, Fe³⁺-EGCG, Al³⁺-EGCG and Zn²⁺-EGCG) yielded strong LMCT bands, while others (Zn²⁺-TA, Zn²⁺-GA, Mn²⁺-GA) featured weaker bands, highlighting the differences in d-d transition strength from the metal and the polyphenol (FIG. 19). The data shows that these assemblies differ significantly in their ligand-metal coordination. We therefore hypothesized that these differences could impact cellular protection capabilities.

### Microbial Coating and Subsequent Viability Assessment

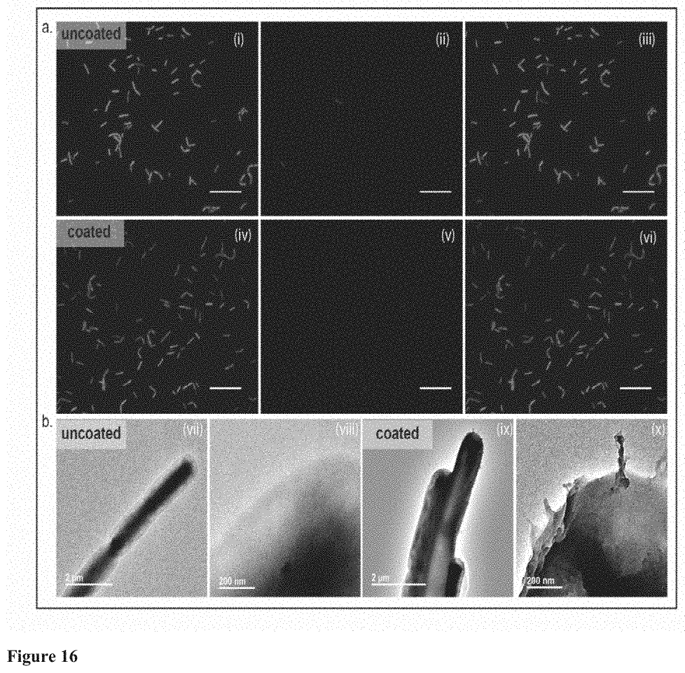

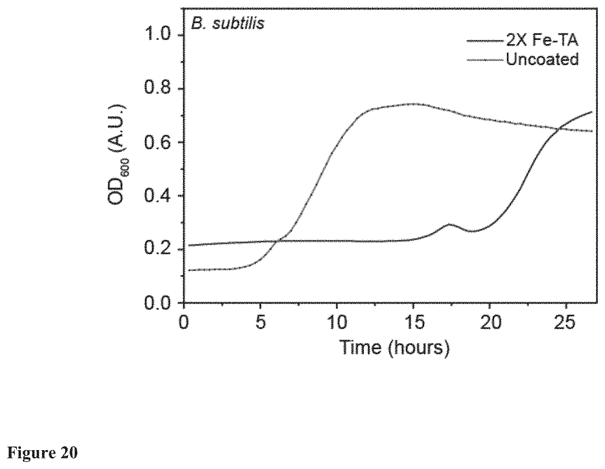

To apply MPNs as protectant agents, these coatings must readily form on microbial substrates with the same ease as the silica particles. Following the protocol detailed in Example 1, Fe³⁺-TA assemblies were readily deposited on the surface of B. subtilis. Surface coating was confirmed through observation of the metals on the surface of the bacteria by transmission electron microscopy (TEM) (FIG. 16b). As can be seen in the image of the uncoated cells, the edges of the uncoated and unstained cells appear smooth and ill-defined. In contrast, the MPNs on the coated cells are visible on cell surface through the sharp contrast observed due to the presence of the metal ions. Following confirmation of coating formation on the cells, we evaluated the impact of the MPNs on cell growth and viability. Bacterial growth was monitored by optical density measurements at 600 nm (OD600), and a delay of 15 hours was observed for the MPN-coated cells to reach exponential phase as compared to the uncoated cells (MPN-coated: 20 hours vs. uncoated controls: 5 hours) (FIG. 20). However, cell viability, which was assessed by LIVE/DEAD staining coupled with fluorescence microscopy, showed minimal bacterial death for MPN-coated B. subtilis (85% viable), as compared to the uncoated control (92% viable) (FIG. 16a). Thus, we believe the lag in exponential phase for the coated cells is due to the rigidity of the coating inhibiting cell division, rather than the MPNs impacting viability.

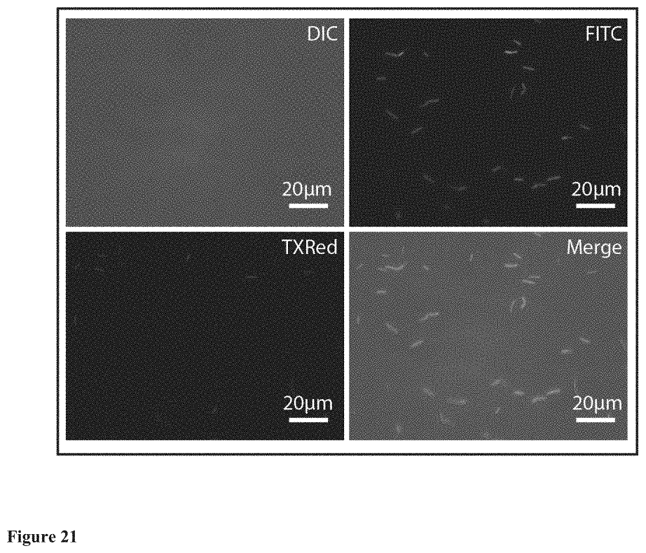

We further observed that a stable MPN assembly helps ensure the biocompatibility of the coating. Without the addition of MOPS buffer at a slightly alkaline pH, which serves to ensure chelation of the polyphenols to the metal ions and rigidify the MPN capsule, a significant loss in the viability of MPN-coated B. subtilis (54% viable) was shown based on the cell viability measurement (FIG. 21). This significant decrease in viability observed in the absence of MOPS buffer addition is attributed to the protonated polyphenols and unchelated metal ions in solution. However, a stable MPN assembly under neutral pH demonstrated high biocompatibility with equivalent viability to uncoated cells. Though simple coating is a first step in the development of non-sporulated B. subtilis for probiotics, lyophilization is helpful for formulation and delivery of these microbes to the gut. Thus, we next investigated the impact of lyophilization on B. subtilis viability.

### Role of Phenol in MPN Microbial Protection

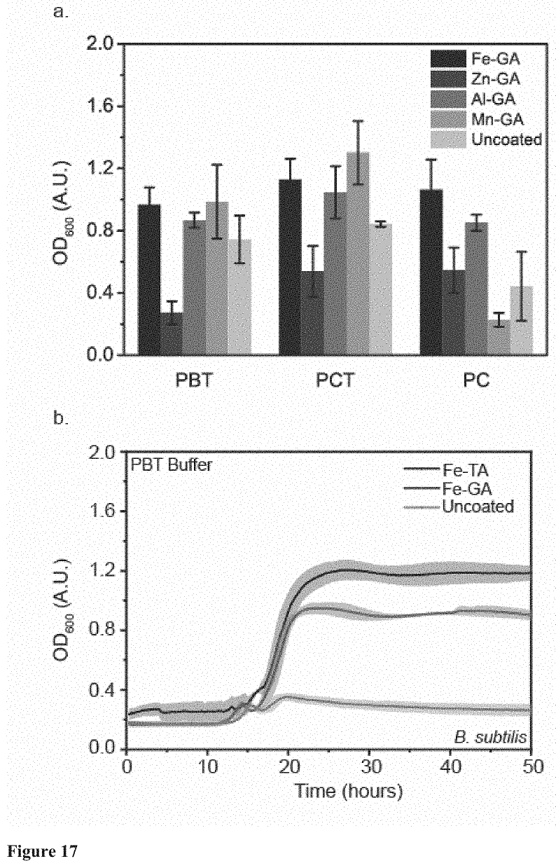

The data in example 1 shows that Fe³⁺-TA MPNs protected living microbes (E. coli and B. thetaiotaomicron) from stresses caused by the freezing and lyophilization processes, significantly increasing their viability post-reconstitution. We hypothesized that this protection could be extended to B. subtilis. The most significant protection in Example 1 is shown with TA-containing MPNs. We therefore began our evaluations of the impact of MPN coating on post-lyophilization viability of B. subtilis with Fe³⁺-TA MPNs. Fe³⁺-TA-coated B. subtilis were flash-frozen and lyophilized in different buffers (phosphate citrate (PC) or phosphate buffered saline (PB) containing 0.1M trehalose (cryoprotectant-PCT or PBT, respectively). OD600 was monitored post-reconstitution in the nutrient broth medium at 30° C. (FIG. 17a). Surprisingly, the protection of cells with this coating was found to be highly buffer-dependent. OD600 values after 48 hours of post-reconstitution growth was fastest when lyophilized in PCT, with high variability in growth from lyophilization in other buffered conditions (FIG. 17a). Thus, we sought to investigate whether altering the components of the MPN impacted post-lyophilization viability. We expanded the polyphenols investigated to include EGCG and GA. No significant difference was observed in the post-reconstitution growth of Fe³⁺-EGCG-coated B. subtilis as compared to the uncoated controls. Yet interestingly, Fe³⁺-GA-coated B. subtilis demonstrated higher OD600 values post-reconstitution as compared to the uncoated controls in both buffers (PBT and PCT).

Though trehalose is canonically used to maintain biomolecule stability during lyophilization, the addition of this molecule is not ideal for the eventual formulation of the cells for oral delivery. Thus, we additionally investigated the impact of MPN coating on protection of B. subtilis following lyophilization in the absence of trehalose. Remarkably, Fe³⁺-GA- and Fe³⁺-TA-coated B. subtilis in cryoprotectant-free buffer (PC) demonstrated 4-fold and 3-fold increases in OD600, respectively. Fe³⁺-GA- and FeIII-TA-coated B. subtilis reach exponential growth within 8 hours, while no growth was observed for either Fe³⁺-EGCG-coated microbes or the uncoated controls (FIG. 17b). These results demonstrate the ability of iron-containing MPNs to provide protection for B. subtilis post-lyophilization even in absence of cryoprotectant. Furthermore, this protection was found to be phenol-dependent, where the efficacy decreased in the order of GA>TA>EGCG. We anticipate that the higher protection afforded by Fe³⁺-GA complexes could be attributed to the modulus of this MPN. Our results demonstrate that the lyophilization buffers impact microbe stabilization during the freeze-drying process. Because of the significant impact varying the polyphenol was found to have on post-lyophilization stability, we further sought to investigate the impact of varying the metal ion in the MPNs.

### Role of Metals on MPN-Based Microbe Protection

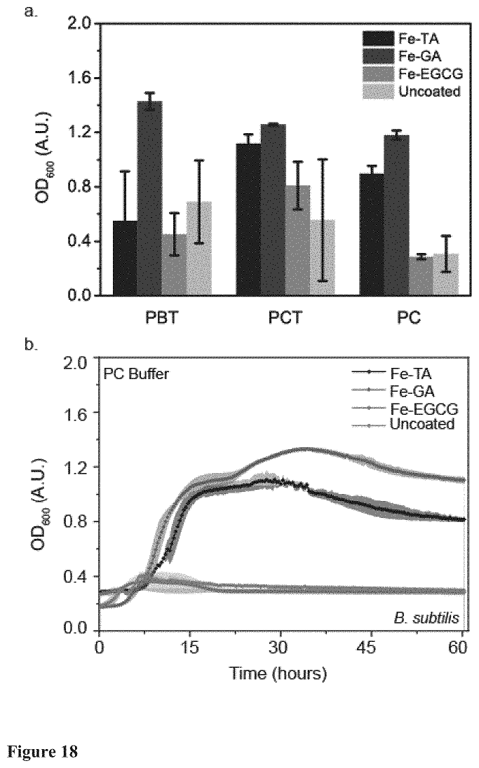

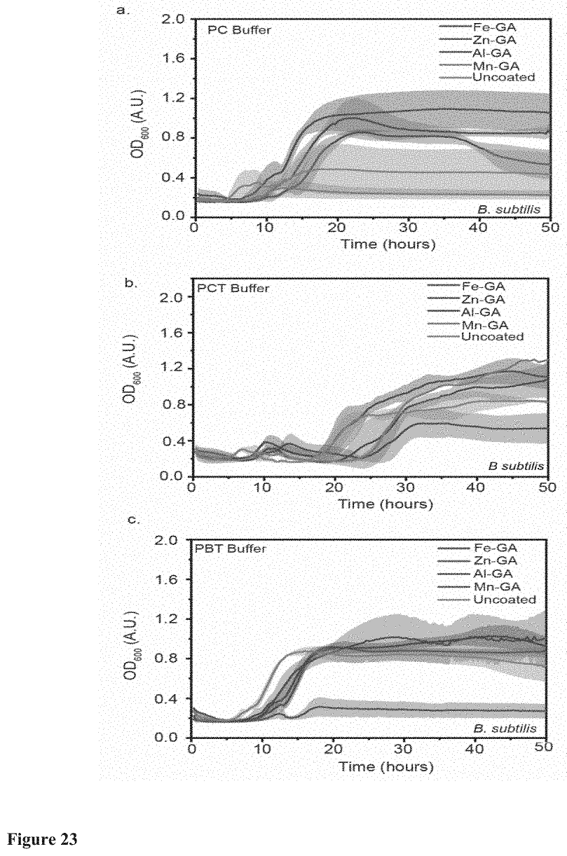

Only biocompatible metal ions were investigated in this study. Specifically, we focused on four ions: Fe³⁺, Mn²⁺, Zn²⁺, and Al³⁺. Because GA demonstrated the highest level of B. subtilis protection of the three polyphenols investigated, the four metal ions were tested as components of MPNs formed with GA. Fe³⁺-GA, Mn²⁺-GA, Zn²⁺-GA, and Al³-GA-coated B. subtilis were flash-frozen and lyophilized in either PBT or PCT (Phosphate Buffer with Trehalose or Phosphate Citrate Buffer with Trehalose). OD600 was monitored after reconstitution in nutrient broth medium (FIG. 18a). No significant difference in OD600 values after 48 h was observed between Fe'-GA, Mn²⁺-GA, and Al³-GA-coated B. subtilis as compared to the uncoated control. Moreover, no growth was observed for Zn²⁺-GA coated microbes. We attribute this to the protective abilities of the trehalose in the buffer because, upon addition of cryoprotectant-free PC buffer, no growth was observed after 48 h for Mn²⁺-GA-coated B. subtilis or the uncoated controls. However, growth was observed for cells coated in MPNs formed with the three other metal ions. Interestingly, the delay in reaching exponential growth (Teg) decreased in the order of Fe³⁺<Al³<Zn²⁺, revealing that metal ions also impact the efficacy of MPN cellular protection (FIG. 23). This suggests that trivalent metal ions increased the network size of MPN assemblies as compared to those using divalent metal ions with the same phenol. Further, the increased recovery rate with Fe³⁺-GA as compared to Al³-GA-coated B. subtilis suggests that the strength of ligand chelation and the size of the metal ionic radius may impact MPN efficacy. Taken together, these results confirm that both the polyphenol and the metal ion used to form MPNs impact the ability of the MPNs to protect B. subtilis following lyophilization.

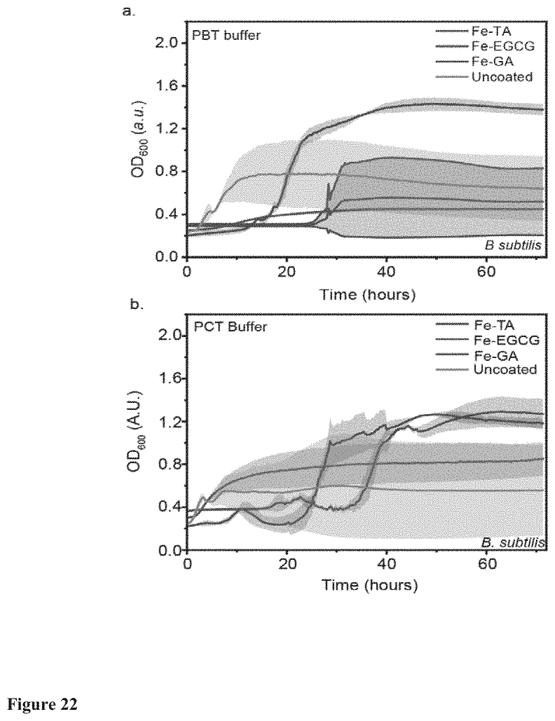

Mild acid treatment leads to faster recovery. Under acidic conditions, hydroxyl groups on the polyphenols in the MPN complexes are protonated, disrupting chelation and leading to disassembly. For oral delivery applications, this pH-dependent degradation is important, as it provides an inherent on-demand disassembly for the cells upon their exposure to stomach acid and intestinal fluid. Though uncoated B. subtilis demonstrated faster recovery than MPN-coated cells post-reconstitution in nutrient broth with PBT or PCT (FIG. 22-23, 25), this appears to be due to the inherent lag in growth consistently observed with MPN-coated B. subtilis (FIG. 20). This hypothesis is supported by the rapid growth of lyophilized MPN-coated B. subtilis when cells are reconstituted in 10 mM L-ascorbic acid solution for 5 min prior to transferal to nutrient broth. The pH of the L-ascorbic acid solution used in this study was measured to be pH=2.5, which is within the range of the pH of stomach acid, which is pH=1.5-3.5. Removal of the coating prior to growth studies decreased the delay time to exponential growth (Teg) of Fe³⁺-GA and Fe³⁺-TA-coated cells dramatically. Further, microbial survival following acid treatment was only observed with MPN-coated cells; no growth after 48 hours was observed with the uncoated controls (FIG. 18). These results further demonstrate the utility of MPN protective coatings for oral delivery of microbes, as these cells will necessarily be exposed to acidic conditions in the stomach and gut.

### Conclusions

We investigated the protection of B. subtilis, an important probiotic strain, from processing stressors during lyophilization. Surprisingly, the efficacy of protection was found to be dependent based on the metal and polyphenol used for assembly. Both the size of the polyphenol and stability of the metal-phenol coordination were factors that influenced their cellular protection; the smallest polyphenol, GA, and the most stable chelated ion, Fe³⁺, were found to provide the highest level of protection. Further, for probiotics and living biotherapeutics, delivery to the gut involves exposure to acidic conditions in the form of stomach acid and intestinal fluid. MPN coatings rapidly disassembled upon mild acid treatment but protected the B. subtilis from the negative impacts of the acid. Overall, B. subtilis is an important probiotic strain currently formulated for delivery as spores, but their ability to germinate in the gut remains debatable. Thus, the ability to deliver viable B. subtilis cells directly is anticipated to improve the efficacy of these strains and serves as an addition study to better understand role of each component in MPNs.

### REFERENCES FOR EXAMPLE 2

(1) Youn, W.; Kim, J. Y.; Park, J.; Kim, N.; Choi, H.; Cho, H.; Choi, I. S. Single-Cell Nanoencapsulation: From Passive to Active Shells. Adv. Mater. 2020, 32 (35).
(2) Guo, Z.; Xie, W.; Lu, J.; Guo, X.; Xu, J.; Xu, W.; Chi, Y.; Takuya, N.; Wu, H.; Zhao, L. Tannic Acid-Based Metal Phenolic Networks for Bio-Applications: A Review. J. Mater. Chem. B 2021, 9 (20), 4098-4110.
(3) Zhang, Z.; Xie, L.; Ju, Y.; Dai, Y. Recent Advances in Metal-Phenolic Networks for Cancer Theranostics. Small 2021, 17 (43), 1-33.
(4) Geng, H.; Zhong, Q.-Z.; Li, J.; Lin, Z.; Cui, J.; Caruso, F.; Hao, J. Metal Ion-Directed Functional Metal-Phenolic Materials. Chem. Rev. 2022.
(5) Wu, D.; Zhou, J.; Creyer, M. N.; Yim, W.; Chen, Z.; Messersmith, P. B.; Jokerst, J. V. Phenolic-Enabled Nanotechnology: Versatile Particle Engineering for Biomedicine. Chem. Soc. Rev. 2021, 50 (7), 4432-4483.
(6) Fan, G.; Cottet, J.; Rodriguez-Otero, M. R.; Wasuwanich, P.; Furst, A. L. Metal-Phenolic Networks as Versatile Coating Materials for Biomedical Applications. ACS Appl. Bio Mater. 2022.
(7) Dai, Q.; Geng, H.; Yu, Q.; Hao, J.; Cui, J. Polyphenol-Based Particles for Theranostics. Theranostics 2019, 9 (11), 3170-3190.
(8) Wu, H. J.; Wu, E. The Role of Gut Microbiota in Immune Homeostasis and Autoimmunity. Gut Microbes 2012, 3 (1).
(9) Vassilev, N.; Vassileva, M.; Martos, V.; del Moral, L. F.; Kowalska, J.; Tylkowski, B.; Malusa, E. Formulation of Microbial Inoculants by Encapsulation in Natural Polysaccharides: Focus on Beneficial Properties of Carrier Additives and Derivatives. Front. Plant Sci. 2020, 11.
(10) Miyamoto-Shinohara, Y.; Imaizumi, T.; Sukenobe, J.; Murakami, Y.; Kawamura, S.; Komatsu, Y. Survival Rate of Microbes after Freeze-Drying and Long-Term Storage. Cryobiology 2000, 41 (3), 251-255.
(11) Minelli, E. B.; Benini, A. Relationship between Number of Bacteria and Their Probiotic Effects. Microb. Ecol. Health Dis. 2008, 20 (4), 180-183.
(12) Fan, G.; Wasuwanich, P.; Rodriguez-Otero, M. R.; Furst, A. L. Protection of Anaerobic Microbes from Processing Stressors Using Metal-Phenolic Networks. J. Am. Chem. Soc. 2022, 144 (6), 2438-2443.
(13) Li, W.; Bing, W.; Huang, S.; Ren, J.; Qu, X. Mussel Byssus-like Reversible Metal-Chelated Supramolecular Complex Used for Dynamic Cellular Surface Engineering and Imaging. Adv. Funct. Mater. 2015, 25 (24), 3775-3784.
(14) Terpou, A.; Papadaki, A.; Lappa, I. K.; Kachrimanidou, V.; Bosnea, L. A.; Kopsahelis, N. Probiotics in Food Systems: Significance and Emerging Strategies Towards Improved Viability and Delivery of Enhanced Beneficial Value. Nutrients. 2019.
(15) Yun, G.; Richardson, J. J.; Biviano, M.; Caruso, F. Tuning the Mechanical Behavior of Metal-Phenolic Networks through Building Block Composition. ACS Appl. Mater. Interfaces 2019, 11 (6), 6404-6410.
(16) Ejima, H.; Richardson, J. J.; Liang, K.; Best, J. P.; van Koeverden, M. P.; Such, G. K.; Cui, J.; Caruso, F. One-Step Assembly of Coordination Complexes for Versatile Film and Particle Engineering. Sci. (New York, N.Y.) 2013, 341 (6142), 154-157.
(17) Wexler, H. M. Bacteroides: The Good, the Bad, and the Nitty-Gritty. Clin. Microbiol. Rev. 2007, 20 (4), 593-621.
(18) Flint, H. J.; Scott, K. P.; Duncan, S. H.; Louis, P.; Forano, E. Microbial Degradation of Complex Carbohydrates in the Gut. Gut Microbes 2012, 3 (4).
(19) Xiaopei, Z.; Amal, A.-D.; Myer, H.; Peter, S.; Jiahe, L.; M., J. P. Applications of Bacillus subtilis Spores in Biotechnology and Advanced Materials. Appl. Environ. Microbiol. 2022, 86 (17), e01096-20.
(20) Rhayat, L.; Maresca, M.; Nicoletti, C.; Perrier, J.; Brinch, K. S.; Christian, S.; Devillard, E.; Eckhardt, E. Effect of Bacillus subtilis Strains on Intestinal Barrier Function and Inflammatory Response. Front. Immunol. 2019, 10.
(21) Colom, J.; Freitas, D.; Simon, A.; Brodkorb, A.; Buckley, M.; Deaton, J.; Winger, A. M. Presence and Germination of the Probiotic Bacillus subtilis DE111® in the Human Small Intestinal Tract: A Randomized, Crossover, Double-Blind, and Placebo-Controlled Study. Front. Microbiol. 2021, 12
(22) Mazaheri, O.; Alivand, M. S.; Zavabeti, A.; Spoljaric, S.; Pan, S.; Chen, D.; Caruso, F.; Suter, H. C.; Mumford, K. A. Assembly of Metal—Phenolic Networks on Water-Soluble Substrates in Nonaqueous Media. Adv. Funct. Mater. 2022, n/a (n/a), 2111942.
(23) Meng, J.; Liu, X.; Niu, C.; Pang, Q.; Li, J.; Liu, F.; Liu, Z.; Mai, L. Advances in Metal-Organic Framework Coatings: Versatile Synthesis and Broad Applications. Chem. Soc. Rev. 2020, 49 (10), 3142-3186.
(24) Geng, H.; Zhuang, L.; Li, M.; Liu, H.; Caruso, F.; Hao, J.; Cui, J. Interfacial Assembly of Metal-Phenolic Networks for Hair Dyeing. ACS Appl. Mater. Interfaces 2020, 12 (26), 29826-29834.
(25) Zhong, Q. Z.; Pan, S.; Rahim, M. A.; Yun, G.; Li, J.; Ju, Y.; Lin, Z.; Han, Y.; Ma, Y.; Richardson, J. J.; et al. Spray Assembly of Metal-Phenolic Networks: Formation, Growth, and Applications. ACS Appl. Mater. Interfaces 2018, 10 (39), 33721-33729.
(26) Guo, J.; Ping, Y.; Ejima, H.; Alt, K.; Meissner, M.; Richardson, J. J.; Yan, Y.; Peter, K.; von Elverfeldt, D.; Hagemeyer, C. E.; et al. Engineering Multifunctional Capsules through the Assembly of Metal-Phenolic Networks. Angew. Chemie Int. Ed. 2014, 53 (22), 5546-5551.
(27) Guo, J.; Richardson, J. J.; Besford, Q. A.; Christofferson, A. J.; Dai, Y.; Ong, C. W.; Tardy, B. L.; Liang, K.; Choi, G. H.; Cui, J.; et al. Influence of Ionic Strength on the Deposition of Metal-Phenolic Networks. Langmuir 2017, 33 (40), 10616-10622.
(28) Sahiner, N.; Sagbas, S.; Sahiner, M.; Silan, C.; Aktas, N.; Turk, M. Biocompatible and Biodegradable Poly(Tannic Acid) Hydrogel with Antimicrobial and Antioxidant Properties. Int. J. Biol. Macromol. 2016, 82, 150-159.
(29) Leslie, S. B.; Israeli, E.; Lighthart, B.; Crowe, J. H.; Crowe, L. M. Trehalose and Sucrose Protect Both Membranes and Proteins in Intact Bacteria during Drying. Appl. Environ. Microbiol. 1995, 61 (10), 3592-3597.
(30) Crowe, L. M.; Womersley, C.; Crowe, J. H.; Reid, D.; Appel, L.; Rudolph, A. Prevention of Fusion and Leakage in Freeze-Dried Liposomes by Carbohydrates. BBA-Biomembr. 1986, 861 (C), 131-140.
(31) Morgan, C. A.; Herman, N.; White, P. A.; Vesey, G. Preservation of Micro-Organisms by Drying; A Review. Journal of Microbiological Methods. Elsevier August 2006, pp 183-193.
(32) Degtyar, E.; Harrington, M. J.; Politi, Y.; Fratzl, P. The Mechanical Role of Metal Ions in Biogenic Protein-Based Materials. Angew. Chemie Int. Ed. 2014, 53 (45), 12026-12044.
(33) Hider, R. C.; Kong, X. Chemistry and Biology of Siderophores. Nat. Prod. Rep. 2010, 27 (5), 637-657.
(34) Shin, J. M.; Choi, G. H.; Song, S. H.; Ko, H.; Lee, E. S.; Lee, J. A.; Yoo, P. J.; Park, J. H. Metal-Phenolic Network-Coated Hyaluronic Acid Nanoparticles for Ph-Responsive Drug Delivery. Pharmaceutics 2019, 11 (12), 1-11.

### Experimental Section

Chemicals and Reagents. All chemicals were reagent grade and used without further purification unless otherwise stated. Tannic acid (99%), gallic acid monohydrate (98%), L-ascorbic acid (99%), iron (III) chloride (FeCl₃, 97%), aluminum chloride (99.9%), zinc sulfate heptahydrate (99%), manganese sulfate monohydrate (99%), 3-(N-morpholino) propanesulfonic acid (MOPS, 99.5%), sodium hydroxide (98%), hydrochloride acid (37%), acetone (99.5%), D-(+)-trehalose dihydrate (99%), sodium phosphate dibasic (99%), sodium phosphate monobasic (99%), and citric acid (99.5%) were purchased from Sigma-Aldrich. Absolute ethanol (molecular biology grade), sodium chloride, and agar (granulated) were purchased from Fisher BioReagents. Nutrient broth (microbiologically tested) powder was purchased from Fluka. Green tea extract (50% EGCG) was purchased from Bulk Supplements. Ultrapure water was generated from an ELGA PURELAB™ Quest UV (Model number: PQDIUVMINSP). LIVE/DEAD™ BacLight™ Bacterial Viability Kit was purchased from ThermoFisher. Nutrient broth liquid media were prepared with 8 g of nutrient broth powder in 1 L of nanopure water (adjust pH to 7.0 with sodium hydroxide) and used after autoclaving (20 min, 121° C.). Nutrient agar plates were prepared on Petri dishes with 20 mL of nutrient agar solution (8 g of nutrient broth powder, and 15 g of agar in 1 L of nanopure water, Adjusting pH to 7.0).

Analysis and Measurement. Optical density or absorbance was measured with a NanoDrop™ One Microvolume UV-Vis Spectrophotometer (Thermo Scientific, USA) or a Biotek synergy mx microplate reader (BioTek Instruments, USA). The cells were observed with a Revolve Fluorescence Microscope (Echo, USA).

Bacterial Strains and Culture. Strain (Bacillus subtilis (Ehrenberg) Cohn (ATCC 6051 LOT: 70044049) was used in this study. Cells were prepared for MPN coating as follows: B. subtilis strains (20% glycerol) stored at −80° C. were streaked onto nutrient agar plates and aerobically kept for —18 h at 30° C. Single colonies were to inoculate nutrient broth. The cultures were kept for —18 h at 30° C. and washed with nanopure water followed by centrifugation (6000×g for 20 min, three times). After the final wash, cells were concentrated to a suspension (OD₆₀₀ of 4.0, 4× stock) and were used immediately.

MPN Encapsulation. MPNs were coated on the surface of B. subtilis as described in Example 1. For different MPNs, equivalent amounts of tannic acid (1.6 mg mL⁻¹), gallic acid (1.5 mg mL⁻¹) or epigallocatechin gallate (2.7 mg mL⁻¹, 50% from tea extract) and an equal volume freshly-made aqueous solution of iron chloride (0.24 mg mL⁻¹), zinc sulfate heptahydrate (0.42 mg mL⁻¹), aluminum chloride (0.19 mg mL⁻¹), or manganese sulfate monohydrate (0.72 mg ml⁻¹) were applied for encapsulation.

Preparation of Lyophilized Cells. The lyophilization of uncoated and MPN-coated B. subtilus were prepared described in Example 1. Before use, the lyophilized cells were reconstituted in sterile 10 mM L-ascorbic acid solution or nutrient broth as necessary.

Bacterial Growth/Viability Assessment. Growth assays were performed in sterile 96-well plates, and OD₆₀₀ was monitored by plate reader. Bacterial viability was determined by live/dead bacterial viability assay. Images were captured using fluorescence microscope for live (green fluorescence: λₑₓ=470/40 nm; λₑₘ=525/50 nm) and dead cells (red fluorescence: λₑₓ=560/40 nm, λₑₘ=630/75 nm) with a 60× oil objective. Cell counts to determine the viable cells percentage were quantified by ImageJ from five randomly-chosen fields of view.

### EXAMPLE 3

Approximately 60,000 new cases of non-muscle invasive bladder cancer (NMIBC) are diagnosed in the United States each year. The standard of treatment for NMIBC is tumor resection followed by immunotherapy with Bacillus Calmette-Guerin (BCG), a Mycobacterium strain. Following BCG treatment, the cure rate for NMIBC is over 90%. Yet, as few as 20% of patients complete the full course due to both lack of patient adherence because of the intensity of the treatment regimen and associated side effects as well as BCG shortages due to manufacturing challenges. For the past decade, Merck has been only approved producer of BCG for distribution in the United States, and their process is highly inefficient and results in very few viable cells per batch, resulting in a severe shortage of this treatment. There is a critical need for new technologies to improve the BCG manufacturing process as well as patient experience and compliance with the regimen. Sufficient access to BCG for NMIBC patients with fewer treatment requirements would improve patient experience and outcomes while decreasing healthcare costs.

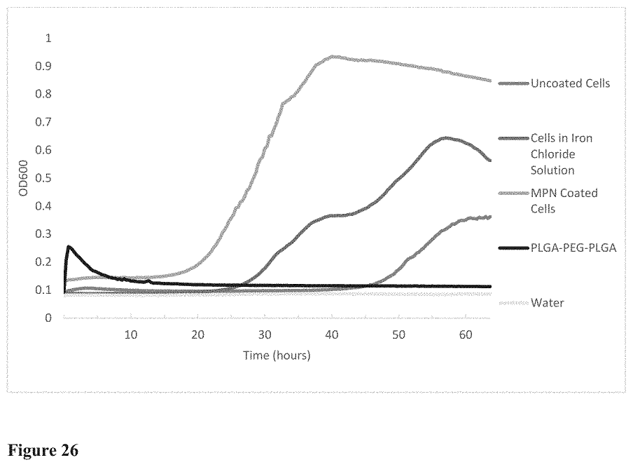

We hypothesize that MPN coatings will protect BCG during freeze-drying and can serve as imaging agents. We successfully coated Mycobacterium smegmatis, a Mycobacterium similar to BCG, with an Fe³⁺ tannic acid (TA) MPN, using the coating techniques described in Examples 1-2. MPN formation on microbes is evidenced by an observable color change due to ligand-to-metal charge transfer (LMCT) in the MPNs (FIG. 26a). Following coating and lyophilization using the coating techniques described in Examples 1-2, reconstituted M. smegmatus recovered significantly faster than the uncoated cells (reaching exponential growth in half of the time of uncoated cells), indicating a higher initial viable cell population in solution (FIG. 26b).

### EXAMPLE 4

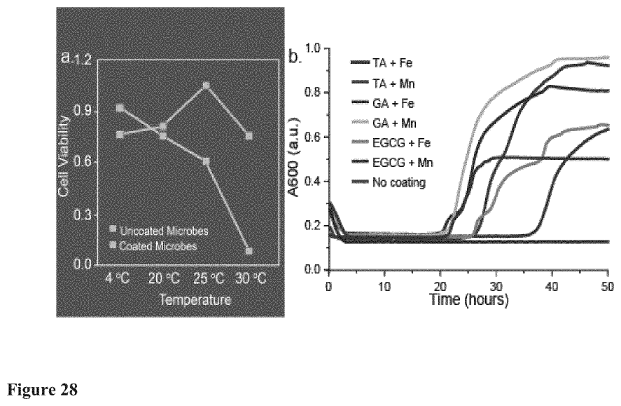

Temperature and humidity can affect cell viability. We evaluated the stability P. chlororaphis having a variety of MPN coatings upon post-lyophilization storage at a range of temperature conditions. The cells were stored aerobically at 4° C., 20° C., 25° C., or 30° C. both for up to 50 hours. Humidity was controlled at relevant temperatures to emulate non-ideal storage conditions using slurries of the proper salts in sealed containers with the samples. The data shows significant survival of these highly temperature-sensitive microbes at elevated temperatures and humidity (FIG. 28, 29), demonstrating the ability of MPNs to protect against stressors.

1. A prokaryotic cell having a coating comprising metal-phenolic networks.

2. The prokaryotic cell of claim 1, wherein the coating comprises a complete coating over the entire cell surface.

3. The prokaryotic cell of claim 1, wherein the coating comprises a single MPN layer, or wherein the coating comprises 2, 3, 4, 5, 6, 7, 8, 9, 10, or more MPN layers.

4. (canceled)

5. The prokaryotic cell of claim 1, wherein
(a) the coating is between about 10 nm and about 100 nm in thickness; and/or
(b) the coating comprises a single metal ion component and a single phenolic component; or the coating comprises more than one metal ion component and/or more than one single phenolic component.

6-7. (canceled)

8. The prokaryotic cell of claim 1, wherein the metal ion component in the MPN comprises one or more cations of aluminum (Al), vanadium (V), chromium (Cr), manganese (Mn), iron (Fe), cobalt (Co), nickel (Ni), copper (Cu), zinc (Zn), zirconium (Zr), molybdenum (Mo), ruthenium (Ru), rhodium (Rh), cadmium (Cd), cerium (Ce), europium (Eu), gadolinium (Gd), terbium (Tb), or combinations thereof, including but not limited to one or more of Fe³⁺, Cr²⁺, Cr³⁺, Cr⁶⁺, Cu⁺, Cu²⁺, Cu³⁺, Cu⁴⁺, Mn²⁺, Mn³⁺, Mn⁴⁺, Mn⁶⁺, Mn⁷⁺, Mo²⁺, Mo³⁺, Mo⁴⁺, Mo⁶⁺, Co²⁺, Ni²⁺, Cd²⁺, Al³⁺, V³⁺, Rh³⁺, Ru³⁺, Zr⁴⁺, Eu³⁺, Gd³⁺, Tb³⁺. and/or Zn²⁺.

9. The prokaryotic cell of claim 1, wherein the metal ion component in the MPN comprises an iron cation, including but not limited to Fe³⁺.

10. (canceled)

11. The prokaryotic cell of claim 1, wherein the polyphenol component of the MPNs comprises one or more of flavonoids (including isoflavonoids and neoflavonoids), tannins, condensed tannins, phenolic acids, catechols, lignans, and stilbenes.

12. The prokaryotic cell of claim 1, wherein the polyphenol component of the MPN comprises tannic acid (TA), gallic acid (GA), epigallocatechin gallate (EGCG), or combinations thereof.

13. The prokaryotic cell of claim 1, wherein
(a) the MPNs comprise an iron cation complexed with one or more of tannic acid (TA), gallic acid (GA), and epigallocatechin gallate (EGCG), or
(b) the MPNs comprise Fe³⁺ complexed with one or more of tannic acid (TA), gallic acid (GA), and epigallocatechin gallate (EGCG).

14. (canceled)

15. The prokaryotic cell of claim 1, wherein
(a) the cell is a bacterial cell or an archaeal cell; and/or
(b) the prokaryotic cell is an anaerobic cell.

16-17. (canceled)

18. The prokaryotic cell of claim 1, wherein the prokaryotic cell is selected from the group consisting of Bacteroides species including but not limited to Bacteroides thetaiotaomicron; any strain belonging to the genus Lactobacillus including but not limited to L. acidophilus, L. crispatus, L. gasseri, group L. delbrueckii, L. salivarius, L. casei, L. paracasei, L. plantarum, L. rhamnosus, L. reuteri, L. brevis, L. buchneri, L. fermentum, L. casei (Gynophilus), L. coleohominis, Lactobacillus delbrueckii subsp. bulgaricus, L. fornicalis, L. gallinarum, Lactobacillus helveticus, Lactobacillus iners, Lactobacillus jensenii, L. mucosae, and L. vaginalis; any strain belonging to the genus Bifidobacterium including but not limited to B. adolescentis, B. angulation, B. bifidum, B. breve, B. catenulatum, B. infantis, B. lactis, B. longum, B. pseudocatenulatum, Bifidobacterium angulatum, Bifidobacterium animalis subsp. lactis, Bifidobacterium dentium, Bifidobacterium magnum; or any strain belonging to S. thermophiles, Pseudomonas fluorescens, Pseudomonas chlororaphis, P. protegens, P. brassicacearum, P. aeruginosa; Azospirillum. brabrasilense, A. lipferum, A. halopraeferens, A. irakense; Acetobacter diazotrophicus; Herbaspirillum seropedicae; Bacillus subtilis, Pseudomonas stutzeri, fluorescens, Pseudomonas chlororaphis, P. putida, P. cepacian, P. vesicularis, P. paucimobilis; Bacillus cereus, B. thuringiensis, B. sphaericus; Shewanella oneidensis; Geobacter bemidjiensis, G. metallireducens, G. sulfurreducens, G. uraniireducens, G. lovleyi; Serratia marcescens, Desulfovibrio vulgaris, D. desulfuricans, Dechloromonas aromatic, Deinococcus radiodurans, Methylibium petroleiphilum, Alcanivorax borkumensis, Archaeglobus fulgidus, Haloferax sp., Halobacterium sp., or from the genera of Akkermansia (including but not limited to Akkermansia muciniphila-probiotic), Anaerostipes, Butyricicoccus, Christensenella, Clostridia, Coprococcus, Dorea, Eubacterium, Faecalibacterium, Cutibacterium, such as Cutibacterium acnes, or Roseburia, Staphylococcus, such as Staphylococcus epidermis or Staphylococcus hominis, Weissella viridescens, or the family Coriobacteriaceae; microbial vaccines Bacille Calmette-Guérin (BCG), Ty21a, Lactobacillus, Lactococcus, Streptococcus, Bacillus, Bifidobacterium, and Vitreoscilla, Pseudomonas, Azospirillum, Azotobacter, Klebsiella, Enterobacter, Alcaligenes, Arthrobacter, Burkholderia, Bacillus, and Serratia; cyanobacteria, Anabaena and Nostoc and genera such as Azotobacter, Beijerinckia, and Clostridium; and Rhizobium, and combinations thereof.

19. The prokaryotic cell of claim 1, wherein
(a) the prokaryotic cell is selected from the group consisting of Bacteroides species including but not limited to Bacteroides thetaiotaomicrons; or
(b) the prokaryotic cell is Bacillus species, including but not limited to Bacillus subtilis, optionally wherein (i) the MPNs comprise Fe³⁺ or Al³⁺, and/or the MPNs comprise Fe³⁺ complexed with GA; or
(c) the prokaryotic cell is Bacillus Calmette-Cuerm (BCG) or Mycobacterium smegmatis, optionally (i) wherein the MPNs comprise Fe³⁺, and/or the MPNs comprise Fe³⁺ complexed with GA.

20-25. (canceled)

26. The prokaryotic cell of claim 1, wherein the cell is lyophilized.

27. The prokaryotic cell of claim 1, further comprising one or more functional groups bound to the MPNs.

28. (canceled)

29. A composition comprising a plurality of the prokaryotic cells of claim 1.

30. The composition of claim 29, wherein the plurality of prokaryotic cells are lyophilized.

31. The composition of claim 30, wherein the composition does not include a cryoprotectant.

32. (canceled)

33. The composition of claim 29, wherein
(a) the formulation comprises beads, powders, capsules, tablets, drops, oil suspensions, gels, lozenges, hydrogels, and/or liquid suspensions; and
(b) the composition is present in a food or beverage product, including but not limited to milk, vogurt, cheese, cream, chocolate, meat, chewing gum, kombucha or other fermentation products, and animal feed or drinking water.

34. (canceled)

35. A method for treating a disorder, comprising administering to a subject in need thereof an amount effective of the composition of claim 29 to treat the disorder.

36-37. (canceled)

38. A method for forming a coating of MPNs on prokaryotic cells, comprising adding metal ions and one or more polyphenols to a suspension of prokaryotic cells, and adjusting pH of the suspension to a final pH of between 7.0 and 8.0 by addition of 3-(N-morpholino) propanesulfonic acid (MOPS) buffer.

39-43. (canceled)
